(19) United States
(12) Patent Application Publication
(10) Pub. No.: US 2015/0253321 A1
(43) Pub. Date: Sep. 10, 2015

(54) Microfluidic Sensors with Enhanced Optical Signals

(71) Applicant: THE TRUSTEES OF THE PRINCETON UNIVERSITY, Princeton, NJ (US)

(72) Inventors: Stephen Y. Chou, Princeton, NJ (US); Chao Wang, Princeton, NJ (US); Ruoming Peng, Princeton, NJ (US)

(73) Assignee: The Turstees of Princeton University, Princeton, NJ (US)

(21) Appl. No.: 14/431,266

(22) PCT Filed: Oct. 1, 2013
(86) PCT No.: PCT/US13/62923

§ 371 Date: Mar. 25, 2015

Related U.S. Application Data
(60) Provisional application No. 61/708,314, filed on Oct. 1, 2012.

(51) Int. Cl. G01N 33/543

(52) U.S. Cl. CPC G01N 33/54386; G01N 2610/00

(57) ABSTRACT

This disclosure provides, among other things, a microfluidic device for detecting an analyte in a liquid, comprising: a substrate; a fluidic channel on a surface of the substrate; and a nanosensor at a location of the channel, the nanosensor comprising: a nanostructure, the nanostructure comprising at least one nanostructure element, each element comprising at least two metallic structures that are separated by a gap, and a capture agent deposited on a surface of the nanostructure, wherein the capture agent specifically binds to the analyte. The nanosensor amplifies a light signal to and/or from the analyte or a light label attached to the analyte, when the analyte is bound or in proximity to the capture agent.

### CROSS-REFERENCING

This application claims the benefit of U.S. provisional application Ser. No. 61/708,314 filed on Oct. 1, 2012, which application is incorporated by reference herein for all purposes.

### STATEMENT REGARDING FEDERALLY SPONSORED RESEARCH

This invention was made with United States government support under Grant No. FA9550-08-1-0222 awarded by the Defense Advanced Research Project Agency (DARPA) The United States government has certain rights in this invention.

### BACKGROUND

There is a great need to enhance a luminescence signal (e.g. a fluorescence signal) and detection sensitivity and reduce the testing time of biological and chemical assays. The application is related to the micro/nanostructures and molecular layers and microfluidic channels and methods for achieving an enhancement (namely amplification of luminescence and improvement of detection sensitivity) and an reduction in assay time, their fabrication and applications.

### SUMMARY

This disclosure provides, among other things, a microfluidic device for detecting an analyte in a liquid, comprising: a substrate; a fluidic channel on a surface of the substrate; and a nanosensor at a location of the channel, the nanosensor comprising: a nanostructure, the nanostructure comprising at least one nanostructure element, each element comprising at least two metallic structures that are separated by a gap, and a capture agent deposited on a surface of the nanostructure, wherein the capture agent specifically binds to the analyte. The nanosensor amplifies a light signal to and/or from the analyte or a light label attached to the analyte, when the analyte is bound or in proximity to the capture agent. This disclosure also provides a portable assay system that can be integrated with a mobile smart phone. The microfluidic device also offers a shorter assay time and smaller sample volume than the devices without microfluidic channels.

### BRIEF DESCRIPTION OF THE DRAWINGS

The skilled artisan will understand that the drawings, described below, are for illustration purposes only. The drawings are not intended to limit the scope of the present teachings in any way. Some of the drawings are not in scale.

FIGS. 1A-1I schematically illustrate various features of some embodiments of a subject nanofluidic device.

FIG. 2 schematically illustrates an exemplary antibody detection assay.

FIG. 3 schematically illustrates an exemplary nucleic acid detection assay.

FIG. 4 schematically illustrates another embodiment nucleic acid detection assay.

FIG. 5 schematically illustrates an exemplary self-assembled monolayer.

FIG. 6 schematically illustrates one embodiment of a system.

FIG. 7 schematically illustrates another embodiment of a system.

FIG. 8 schematically a smartphone embodiment.

FIG. 9. Device structure and fabrication process. (a-b) Device architecture; (c) SEM image of Cr dots array fabricated by nanoimprint; (d-e) bottom-sensor and channel layer fabrication; (f) middle PDMS inlet-and-outlet layer; (g) Au evaporation and bonding of all three layers.

FIG. 10. (a) Optical setup of the model immunoassay experiment. The laser beam scan area is 100 μm by 100 μm; (b)-(c) Fluorescence Intensity versus Concentration. The five parameters logistic regression model shows a detection limit of 850 aM for the D2PA in microfluidic channel device; 2 nM for the glass reference and 1 fM for the D2PA in 96-well plate assay.

FIGS. 11A and 11B. Schematics of NIL patterning of Au nano-dots in fluidic channels. (a) Fused silica substrate coated with bottom stack of SiO₂/ARC; (b) Micro-fluidic channels defined by photolithography; (c) Micro-channels patterned in bottom SiO₂/ARC layers and fused silica, with photoresist stripped; (d) Middle stack of SiO₂/ARC coated on the substrate; (e) Second photolithography to define the nano-feature patterning window for Au nano-dots; (f) Nano-feature patterning window transferred to middle stack SiO₂/ARC by RIE; (g) Imprint resist coated and planarized on the substrate; (h) Nano-holes patterned in imprint resist by a nano-pillar mold and covered with a Cr mask. (i) Au nano-dots patterned in fluidic channels by evaporation and liftoff.

FIG. 12A. Fluorescence enhancement of DNA molecules. (a) Diagrams of fluorescence enhancement by plasmonic nanostructures to achieve enhanced fluorophore excitation and radiative emission. (b) Schematics of simultaneous DNA stretching and fluorescence enhancement by plasmonic D2PA structures in fluidic channels, with the round dots indicating the hot-spots for strong fluorescence enhancement.

FIG. 12B. Schematics of patterning plasmonic Au D2PA array in fluidic channels. a-b, The designed cross-sectional geometrical dimension of D2PA arrays in channels: (a) before sealing; (b) after bonding for DNA fluorescence enhancement. c-j, The fabrication schematics: (c) Fused silica substrate coated with bottom stack of SiO₂/ARC. (d) Micro-fluidic channels defined in fused silica. (e) Coating middle stack of SiO₂/ARC. (f) Second photolithography and RIE to define the D2PA-patterning window in SiO₂ (middle stack). (g) Nano-holes UV-imprinted in resist. (h) Cr nano-dots patterned on fused silica after liftoff. (i) Nano-pillar etching in fused silica. (j) D2PA arrays after Au evaporation.

FIG. 12C. Fabricated nano-pillars and D2PA nano-structures in fluidic channels. a-b, Side-view (45°) SEM images of nano-pillars selective patterned in fluidic channels and self-aligned to channel edge with the dimensions as 60 nm in height, 115 in diameter. (c) Optical images of nano-pillar region in channels connected to inlet/outlet and accessory channels. d-e, SEM images of fabricated D2PA arrays: (d) high-magnification 45° side-view of 145 nm diameter D2PA arrays with a vertical cavity gap of 10 nm, with cross-sectional view shown as insert; and (e) low-magnification top view to show large-area uniform patterning. (f) Optical images of D2PA arrays in fluidic channels after Au deposition.

Corresponding reference numerals indicate corresponding parts throughout the several figures of the drawings. It is to be understood that the drawings are for illustrating the concepts set forth in the present disclosure and are not to scale.

Before any embodiments of the invention are explained in detail, it is to be understood that the invention is not limited in its application to the details of construction and the arrangement of components set forth in the following description or illustrated in the drawings.

### DEFINITIONS

Before describing exemplary embodiments in greater detail, the following definitions are set forth to illustrate and define the meaning and scope of the terms used in the description.

The term “molecular adhesion layer” or “adhesion/spacer layer” refers to a layer or multilayer of molecules of defined thickness that comprises an inner surface that is attached to the nanosensors and an outer (exterior) surface can be bound to capture agents. It also controls the distance from the metal to the molecules or materials that emitting light.

The term “capture agent-reactive group” refers to a moiety of chemical function in a molecule that is reactive with capture agents, i.e., can react with a moiety (e.g., a hydroxyl, sulfhydryl, carboxy or amine group) in a capture agent to produce a stable strong, e.g., covalent bond.

The term “capture agent” as used herein refers to an agent that binds to a target analyte through an interaction that is sufficient to permit the agent to bind and concentrate the target molecule from a heterogeneous mixture of different molecules. The binding interaction is typically mediated by an affinity region of the capture agent. Typical capture agents include any moiety that can specifically bind to a target analyte. Certain capture agents specifically bind a target molecule with a dissociation constant (K_{D}) of less than about 10⁻⁶ M (e.g., less than about 10⁻⁷M, less than about 10⁻⁸M, less than about 10⁻⁹M, less than about 10⁻¹⁰ M, less than about 10⁻¹¹ M, less than about 10⁻¹² M, to as low as 10⁻¹⁶ M) without significantly binding to other molecules. Exemplary capture agents include proteins (e.g., antibodies), and nucleic acids (e.g., oligonucleotides, DNA, RNA including tamers).

The terms “specific binding” and “selective binding” refer to the ability of a capture agent to preferentially bind to a particular target molecule that is present in a heterogeneous mixture of different target molecule. A specific or selective binding interaction will discriminate between desirable (e.g., active) and undesirable (e.g., inactive) target molecules in a sample, typically more than about 10 to 100-fold or more (e.g., more than about 1000- or 10,000-fold).

The term “protein” refers to a polymeric form of amino acids of any length, i.e. greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc, and polypeptides with secondary or tertiary structure, and polypeptides that are strongly bound, e.g., covalently or non-covalently, to other moieties, e.g., other polypeptides, atoms, cofactors, etc.

The term “antibody” is intended to refer to an immunoglobulin or any fragment thereof, including single chain antibodies that are capable of antigen binding and phage display antibodies).

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The term “complementary” as used herein refers to a nucleotide sequence that base-pairs by hydrogen bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. Typically, “complementary” refers to a nucleotide sequence that is fully complementary to a target of interest such that every nucleotide in the sequence is complementary to every nucleotide in the target nucleic acid in the corresponding positions. When a nucleotide sequence is not fully complementary (100% complementary) to a non-target sequence but still may base pair to the non-target sequence due to complementarity of certain stretches of nucleotide sequence to the non-target sequence, percent complementarily may be calculated to assess the possibility of a non-specific (off-target) binding. In general, a complementary of 50% or less does not lead to non-specific binding. In addition, a complementary of 70% or less may not lead to non-specific binding under stringent hybridization conditions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 200 nucleotides and up to 300 nucleotides in length, or longer, e.g., up to 500 nt in length or longer. Oligonucleotides may be synthetic and, in certain embodiments, are less than 300 nucleotides in length.

The term “attaching” as used herein refers to the strong, e.g, covalent or non-covalent, bond joining of one molecule to another.

The term “surface attached” as used herein refers to a molecule that is strongly attached to a surface.

The term “sample” as used herein relates to a material or mixture of materials containing one or more analytes of interest. In particular embodiments, the sample may be obtained from a biological sample such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In particular embodiments, a sample may be obtained from a subject, e.g., a human, and it may be processed prior to use in the subject assay. For example, prior to analysis, the protein/nucleic acid may be extracted from a tissue sample prior to use, methods for which are known. In particular embodiments, the sample may be a clinical sample, e.g., a sample collected from a patient.

The term “analyte” refers to a molecule (e.g., a protein, nucleic acid, polymer or other molecule, complex of the same or particle) that can be bound by a capture agent and detected.

The term “assaying” refers to testing a sample to detect the presence and/or abundance of an analyte.

As used herein, the terms “determining,” “measuring,” and “assessing,” and “assaying” are used interchangeably and include both quantitative and qualitative determinations.

As used herein, the term “light-emitting label” refers to a label that can emit light when under an external excitation. This can be luminescence. Fluorescent labels (which include dye molecules or quantum dots), and luminescent labels (e.g., electro- or chemi-luminescent labels) are types of light-emitting label. The external excitation is light (photons) for fluorescence, electrical current for electroluminescence and chemical reaction for chemi-luminscence. An external excitation can be a combination of the above.

The phrase “labeled analyte” refers to an analyte that is detectably labeled with a light emitting label such that the analyte can be detected by assessing the presence of the label. A labeled analyte may be labeled directly (i.e., the analyte itself may be directly conjugated to a label, e.g., via a strong bond, e.g., a covalent or non-covalent bond), or a labeled analyte may be labeled indirectly (i.e., the analyte is bound by a secondary capture agent that is directly labeled).

The term “hybridization” refers to the specific binding of a nucleic acid to a complementary nucleic acid via Watson-Crick base pairing. Accordingly, the term “in situ hybridization” refers to specific binding of a nucleic acid to a metaphase or interphase chromosome.

The terms “hybridizing” and “binding”, with respect to nucleic acids, are used interchangeably.

The term “capture agent/analyte complex” is a complex that results from the specific binding of a capture agent with an analyte. A capture agent and an analyte for the capture agent will usually specifically bind to each other under “specific binding conditions” or “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens and nucleic acid hybridization are well known in the art (see, e.g., Harlow and Lane (Antibodies: A Laboratory Manual Cold Spring Harbor Laboratory, Cold Spring Harbor, N.Y. (1989) and Ausubel, et al, Short Protocols in Molecular Biology, 5th ed., Wiley & Sons, 2002).

The term “specific binding conditions” as used herein refers to conditions that produce nucleic acid duplexes or protein/protein (e.g., antibody/antigen) complexes that contain pairs of molecules that specifically bind to one another, while, at the same time, disfavor to the formation of complexes between molecules that do not specifically bind to one another. Specific binding conditions are the summation or combination (totality) of both hybridization and wash conditions, and may include a wash and blocking steps, if necessary.

For nucleic acid hybridization, specific binding conditions can be achieved by incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C.

For binding of an antibody to an antigen, specific binding conditions can be achieved by blocking a substrate containing antibodies in blocking solution (e.g., PBS with 3% BSA or non-fat milk), followed by incubation with a sample containing analytes in diluted blocking buffer. After this incubation, the substrate is washed in washing solution (e.g. PBS+TWEEN 20) and incubated with a secondary capture antibody (detection antibody, which recognizes a second site in the antigen). The secondary capture antibody may conjugated with an optical detectable label, e.g., a fluorophore such as IRDye800CW, Alexa 790, Dylight 800. After another wash, the presence of the bound secondary capture antibody may be detected. One of skill in the art would be knowledgeable as to the parameters that can be modified to increase the signal detected and to reduce the background noise.

The term “a secondary capture agent” which can also be referred to as a “detection agent” refers a group of biomolecules or chemical compounds that have highly specific affinity to the antigen. The secondary capture agent can be strongly linked to an optical detectable label, e.g., enzyme, fluorescence label, or can itself be detected by another detection agent that is linked to an optical detectable label through bioconjugation (Hermanson, “Bioconjugate Techniques” Academic Press, 2nd Ed., 2008).

The term “biotin moiety” refers to an affinity agent that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10-8M. A biotin affinity agent may also include a linker, e.g., -LC-biotin, -LC-LC-Biotin, -SLC-Biotin or -PEGn-Biotin where n is 3-12.

The term “streptavidin” refers to both streptavidin and avidin, as well as any variants thereof that bind to biotin with high affinity.

The term “marker” refers to an analyte whose presence or abundance in a biological sample is correlated with a disease or condition.

The term “bond” includes covalent and non-covalent bonds, including hydrogen bonds, ionic bonds and bonds produced by van der Waal forces.

The term “amplify” refers to an increase in the magnitude of a signal, e.g., at least a 10-fold increase, at least a 100-fold increase at least a 1,000-fold increase, at least a 10,000-fold increase, or at least a 100,000-fold increase in a signal.

The word “detecting” means detecting whether something is present or not, as well as quantitatively measuring the amount of something to provide an absolute or relative value, e.g., a value relative to a control analyte.

The words “a” and “an” mean one or a plurality (i.e., “at least one”) unless otherwise indicated, e.g., by using the word “single”.

The term “on a surface of the substrate” means etched into a surface of a substrate, as well as fabricated onto a surface of a substrate.

The term “lift-off process” means the process where a dissolvable material layer is deposited on a substrate surface, the material has an opening that exposes a part of the substrate, then a unsolvable material is deposited on top of the dissolvable material as well as inside the opening and on the substrate, but a little on the opening sidewall, a solvent remove the dissolvable materials and the unsolvable material on top but the unsolvable material on the substrate.

The following pairs of terms are interchangeable and identify the same subject: “liquid” and “liquid sample”; “protrusion” and “pillar”; “protrusion height” and “pillar height”; “cap” and “disk (disc)”. Other pairs of terms that refer to the same thing may be found in this disclosure.

The term “layer” means a thin layer of continuous or discontinuous film. The film may be a polarity of discrete elements or molecules, a molecular monolayer, and may cover only a portion of surface or only a particular type of material (e.g. metal not dielectrics).

It must be noted that as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, and reference to “a detection agent” includes a single detection agent and multiple detection agents.

### DETAILED DESCRIPTION OF EXEMPLARY EMBODIMENTS

The following detailed description illustrates some embodiments of the invention by way of example and not by way of limitation.

The invention is related to the structures, components, systems, methods, fabrications, and applications of a microfluidic device and an associated system that can enhance sensing (either detecting and/or quantifying), using optical means, at least an analyte in a liquid. The analyte include various biological and chemical materials. The microfluidic device in the invention is termed “microfluidic sensor enhanced by coupled-metallic nanostructures” (MOSEC).

More specifically, the invention is related to the enhancement that include an increase in sensing sensitivity, a reduction of sensing time and/or sensing cost, a simplification of sensing, multiplexing of sensing, ease of use, and usefulness. In certain embodiments, the system may communicate with a smartphone in order to facilitate communication between a user and a health care professional, e.g., a doctor or a clinician or nurse, etc.

The invention is also related to the enhancement of an assay's detection sensitivity and speed as well as reduction device size and test sample volume by using metallic and dielectric nanostructures, molecular binding (immuoassays and nucleic acid assays), and microfluidic channels.

The invention is also related to use of metallic nanostructures (also called plasmonic nanostructure) and dielectric nanostructures amplify (enhance) an optical signal (also term “light signal”) to and from a material placed on the nanostructures or in proximity of the nanostructures.

The invention is also related to detection of an optical signal, including fluorescence, luminescence, and Raman signal using light source, optical systems, and optical detectors.

Some aspects of the metallic nanostructures and the molecular binding process used in the nanosensor of the MOSEC's sensing element without microfluidic channel have been disclosed in WO2012/024006, which is incorporated by reference herein. However, by new designs that drastically reduces the size and form of the nanosensors, that integrates them with microfluidic channels, that integrates multiple nanosensors and multiple microfluidic channels on a single chip, and that integrates with mobile smartphone, etc., as disclosed in this invention, offer many novel functional and utility advantages over the just the nanosensor alone. The advantages include (a) small detector form factors for nanodetecters, assays, and the entire systems (including optical excitation and detection), (b) lower noise and better signal due to the integration; (c) small sample size; (d) assay multiplexing in detection multiple analytes in a liquid in a single test run; (e) much shorter assay testing time, (f) much lower cost, can be fabricated monlithocally to further reduce the cost; (g) convenient to use, (h) much more accessible by people; (i) used for personal health care, (j) use with mobile smart phone, and (k) the test results being transmitted to professional personal or professional data base quickly.

### Microfluidic Devices

With reference to the figures, disclosed herein is microfluidic device 100 for detecting and/or quantifying at least one type of analyte in a liquid comprises a substrate 110, a fluidic channel 120 in the substrate or on a surface of the substrate; a nanosensor 130 at a location of the channel. The nanosensor 130 comprises one nanostructure layer 132 and a molecular adhesion layer and attachment of capture agents combination 134 that is deposited on a surface of the nanostructure 132. The nanostructure layer 132 comprises one or a plurality of elements, each of the elements comprising of at least two metallic structures separated by a gap. The capture agent specifically binds to the target analyte to be detected and/or quantified. The nanosensor 130 of the microfluidic device 100 amplifies a light signal to or from the analyte or both, or to or from a light signal by a light label attached to the analyte or both, when the analyte is bound or in proximity to the capture agent 134. The light signal is related to a property of the analyte and/or the light signal by a light label. The light signal can be various luminescence (e.g., chemiluminescent or electroluminescent, or fluorescence (photoluminences)), or surface enhanced Raman scattering (SERS).

The microfluidic device 100 amplifies further comprises an inlet 150 for a liquid to flow into the fluidic channel 130 and, as an option, filters and separators 140, waste dump 160 for liquid, and an optional outlet 170 for a liquid.

The light amplification comes from one or several following factors: the nanosensor can (a) absorb light excitation effectively (e.g. the light at a wavelength that excites fluorescent moieties), (b) focus the absorbed light into certain locations, (c) place the analytes into the regions where most of light are focused, and (d) radiate efficiently the light generated by analytes from the locations where the analytes immobilized.

In some embodiments, different capture agents are attached to the nanosensor surface with each capture agent coated on a different location of the surface, e.g., in the form of an array, hence providing multiplexing in detections of different analysts, since each location is specific for capturing a specific kind of analyte.

In some embodiments, a subject nanosensor may further comprise labeled analyte that is specifically bound to the capture agent. As noted above, the labeled analyte may be directly or indirectly labeled with a light-emitting label. In embodiments in which an analyte is indirectly labeled with a light-emitting label, the analyte may be bound to a second capture agent, also termed: detection agent (e.g., a secondary antibody or another nucleic acid) that is itself optically labeled. The second capture agent may be referred to as a “detection agent” in some cases. In some embodiments, the thickness of the molecular adhesion layer and the capture agent layer is selected to optimize the amplification of the light signal.

In some embodiments the surface of the substrate may be hydrophobic or hydrophilic, as desired. In some cases, surface of the substrate may be made of any suitable plastic, e.g., cyclic olefin copolymer (COC), poly(methyl methacrylate) (PMMA), polycarbonate (PC), cyclic olefin polymer (COP), liquid crystalline polymer (LCP), polyamide (PA), polyethylene (PE), polyimide (PI), polypropylene (PP), poly(phenylene ether) (PPE), polystyrene (PS), polyoxymethylene (POM), polyether ether ketone (PEEK), polyether sulfone (PES), poly(ethylene phthalate) (PET), polytetrafluoroethylene (PTFE), polyvinyl chloride (PVC), polyvinylidene fluoride (PVDF), polybutylene terephthalate (PBT), fluorinated ethylene propylene (FEP), perfluoroalkoxyalkane (PFA) and polydimethylsiloxane (PDMS). A key function of the substrate is to provide mechanical support; while the other functions of the substrate are transmitting and/or manipulating light, different properties of hydrophobic and hydrophilic surfaces, control of thermal conductions and others. The material can be any suitable material.

### Fluidic Channels

The fluidic channel 130 is for flowing liquid that are test samples, test reagents (such as detection proteins, detection nucleic acids, light labels, solvents, blocking solutions, etc). The microfluidic device 100 can have multiple fluidic channels 130. Each of the fluidic channels can have zero, one, and more than one nanosensors 130. The fluidic channel can be oriented in different directions and can across each other for the sensing need. The microfluidic device reliably performs basic functions such as transportation, mixing, or separation of a fluid in a quantity desired by a user, by using a valve or a pump system. To operate the valve or pump system of the microfluidic device, thermal, magnetic, electrical, or pneumatic methods are used.

The inlet 150 is for a liquid to enter a fluidic channel.

The filters and separators 140, which an optional, are for separating the targeted analyzes from the materials that could interfere the sensing.

The waste dump 160, which is also optional, is for storing the wastes in the sensing.

The outlet 170, which is also optional, is for removing the waste and facilitates the flow of the liquid in the fluidic channel(s). The facilitation includes the control of the liquid flow speed.

The microfluidic channel can be in a closed form where all walls of the channel is closed, (termed “closed channel”); or an open form where one of the channel walls is open (termed “open wall channel”) where the capillary force keeps a fluid in an open channel.

The channels may be of any suitable width and depth. To reduce an assay time the channel height, defined as the distance between a nanosensor surface to its ceiling (i.e. opposite wall), should be small. This is because that an analyte moves in a liquid by diffusion, and it would take time for an analyte to diffuse from a location in a liquid to a capture agent immobilized on a channel wall. The time is often proposal to the square of the distance. Reducing the distance can drastically reduce the time of capturing the analyte by immobilized capture agent and hence the assay time. For short assay time, a preferred channel height is from 2 nm to 50 micrometer (μm), though the channel height less 100 μm, 200 μm, and 500 μm should also have advantages over conventional 96 wall plates in fast assay time. In the other worlds, the microfluidic channel should have a cross-section such that the total fluid thickness on the top surface of the nanosensor is in the range of 2 nm to 50, or if a longer assay time can be tolerated, less than 500 micron. Both the height and the width of the channel can affect the flow speed of a liquid, and should be optimized for a desired flow speed. Often the channel height is prefixed by the analyte capture time, then one can use the channel width to adjusting the liquid flow speed.

The embodiments of the fluidic channels and associated elements include but not limited to the following selections. One channel contains more than one nanosensors, each sensor is at a different location of the channel and may be coated with a different capture agent to detect a different kind of analyte. Multiple channels are used, each of them have zero, one, or more than on nanosensors. Additional fluidic channels are used to introduce different reagents and solvents. The channels can be oriented in different directions on the substrate surface and/or intersect, the intersection may cause two channels connected, or the two channel can across without being connected. The number of channels and nanosensors are related to the multiplexing of detection of different analytes. The liquid and reagents can flow in the multi-channel in parallel or sequentially.

The number of microfluidic channels, the number of nanosensors for a given microfluidic channel, and the number of inlets, outlets, waste dumps, filters, separators, and on-chip fluidic controls depending upon the needs of sensing and the number of analytes to be sensed.

In some multi-channel microfluidic devices, fluidic control devices on the microfluidic device 100 are needed, such devices have been described in, e.g., US20130244906, US20130244337, US20130244270, US20130240073, US20130239082, US20130236375 and US20130230906, which publications are incorporated by reference.

### Nanosensors

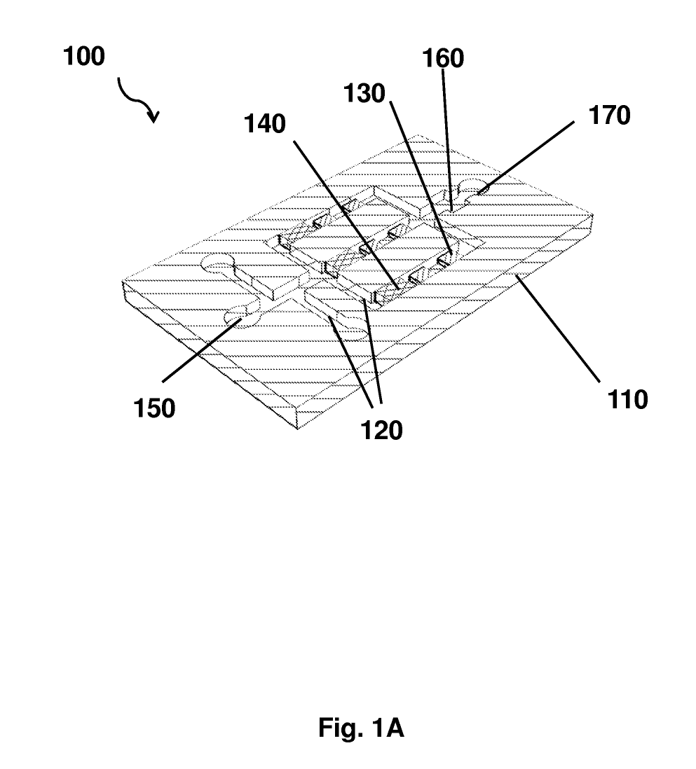

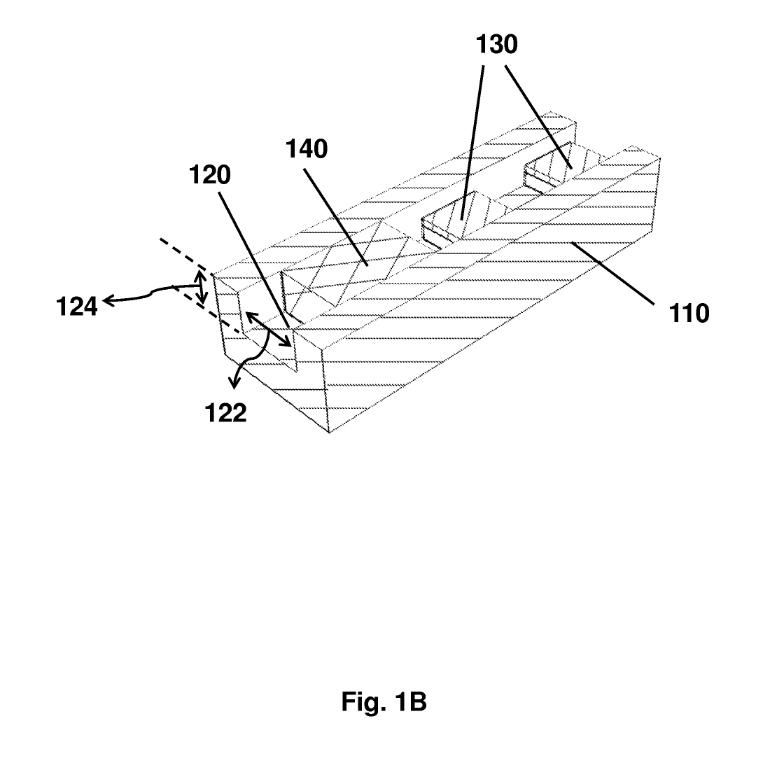

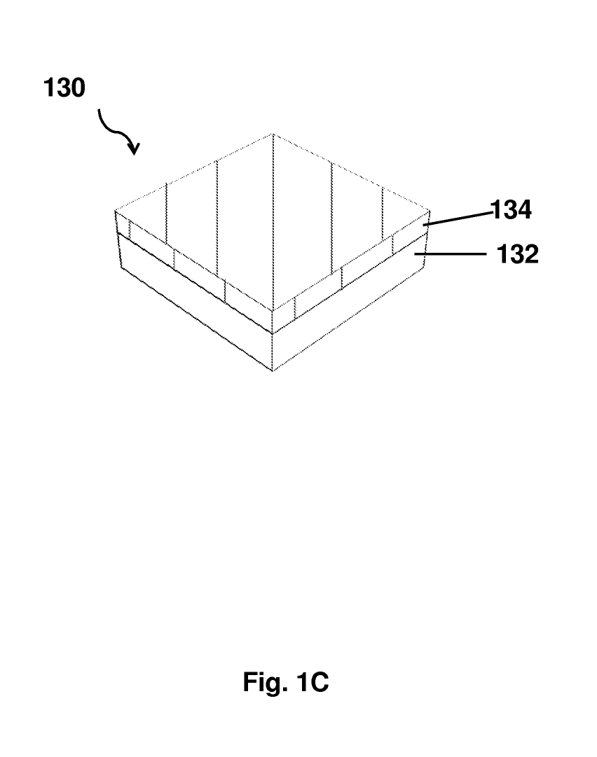

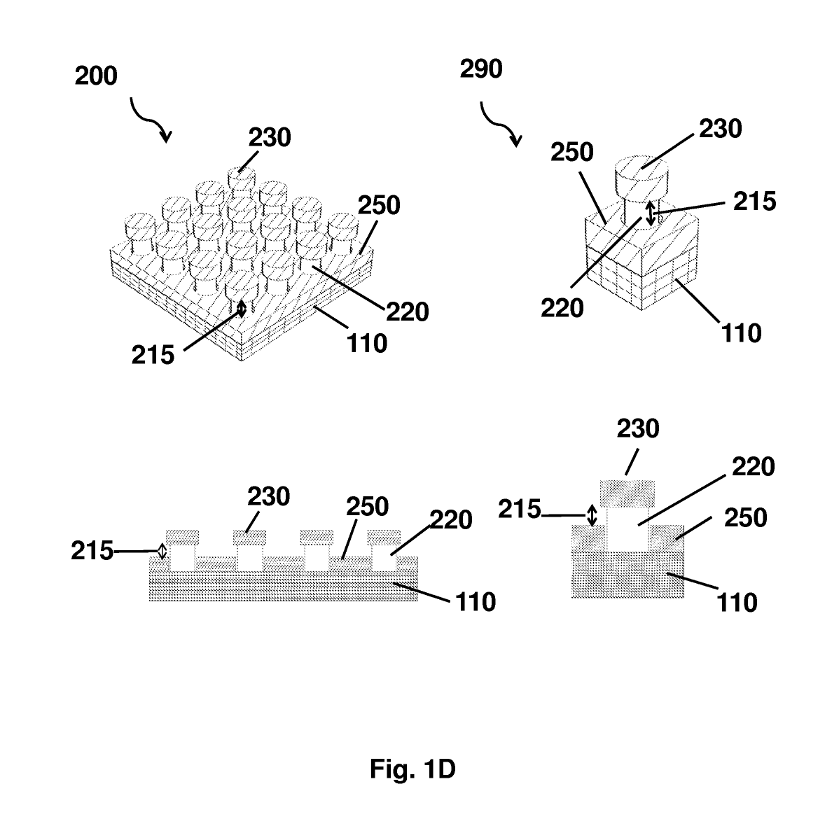

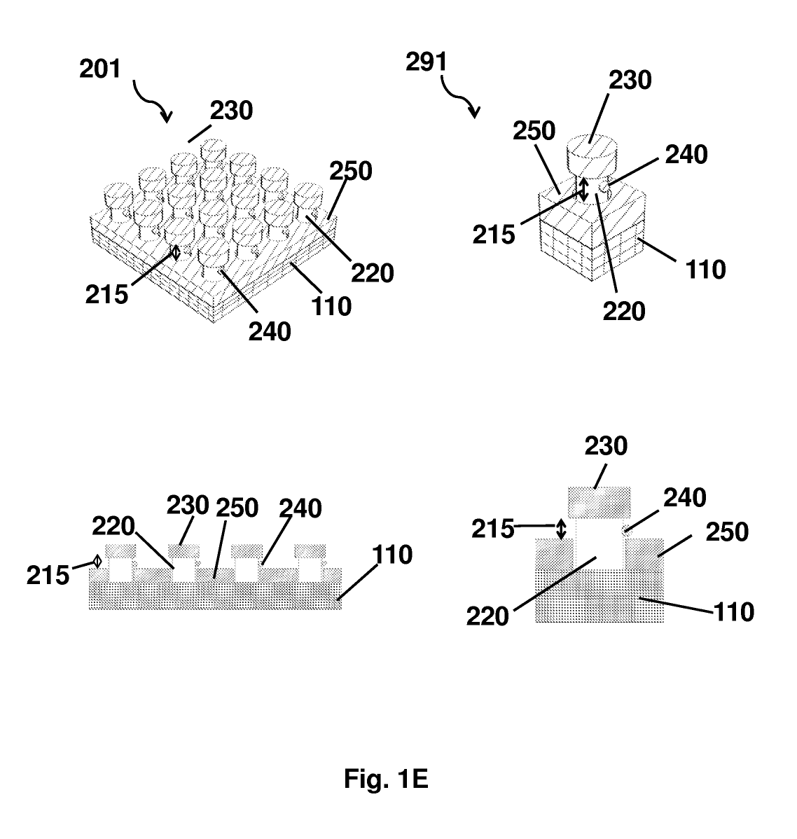

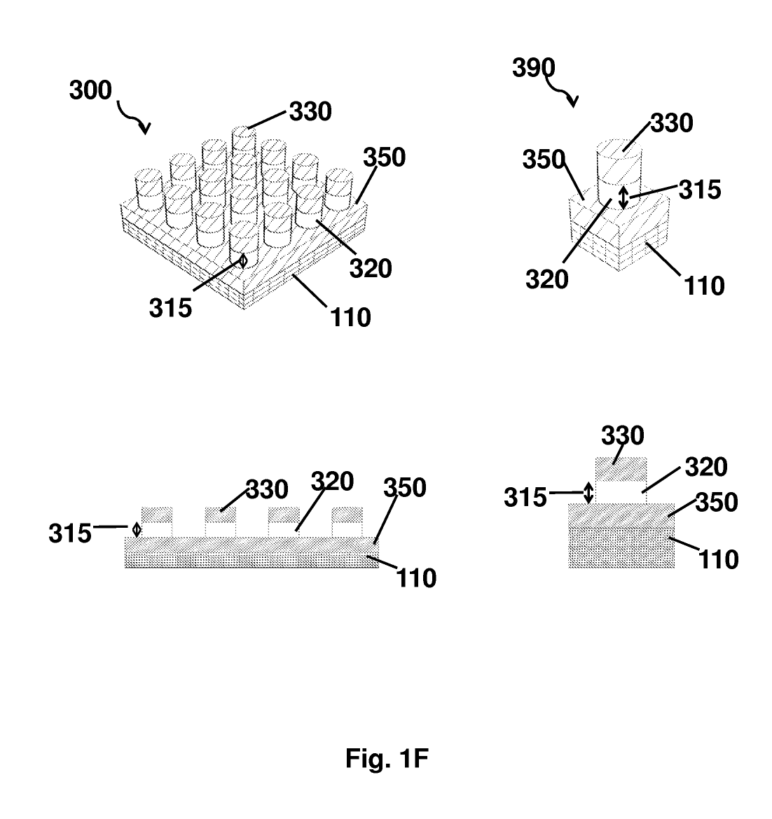

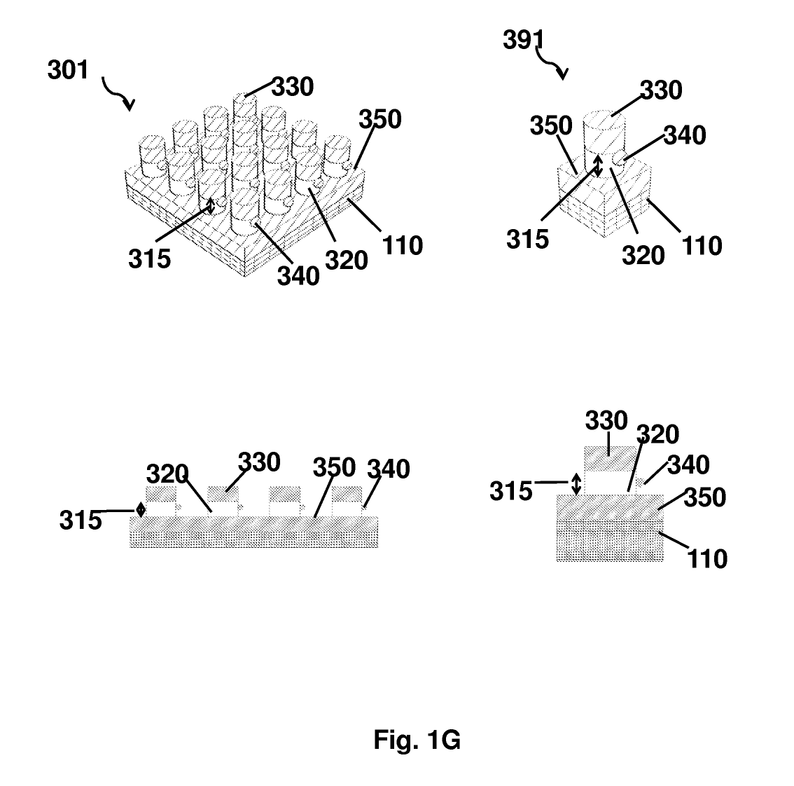

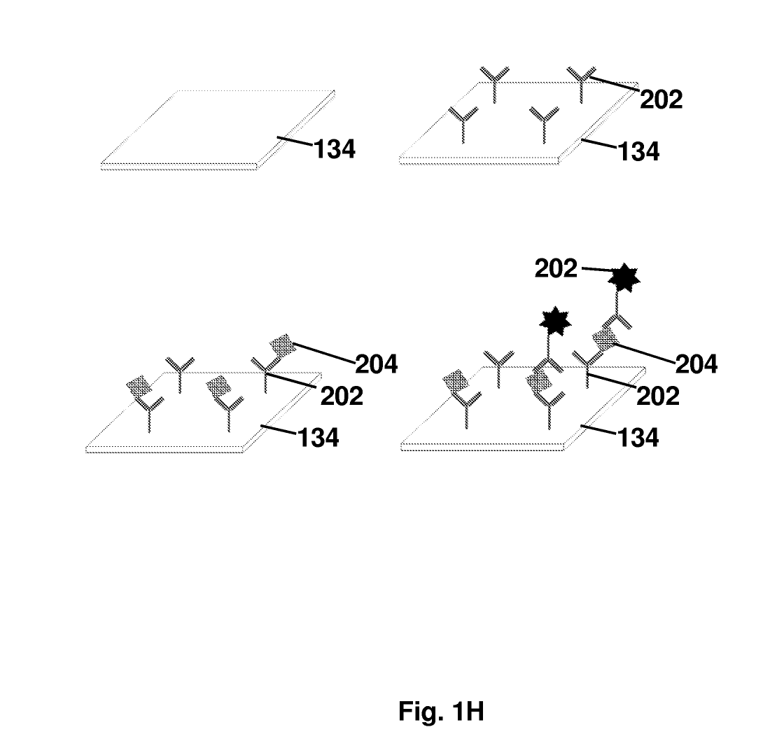

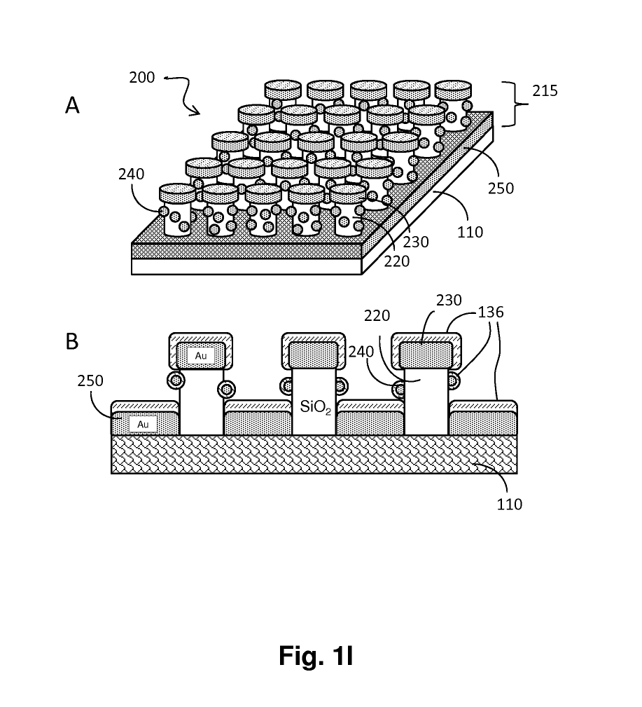

With reference to the figures, particular FIG. 1C, 1H, 1I, 2-4, a nanosensor 130 comprises a nanostructure layer 132 and a molecular adhesion layer and attachment of capture agents combination 134 that is deposited on a surface of the nanostructure 132.

The a molecular adhesion layer and attachment of capture agents combination 134 is often in the form discontinuous film, can be a molecular monolayer, and may cover only a portion of the nanostructure 132 (e.g. only the metal of the nanostructure layer). In some embodiments, the molecular adhesion layer and attachment of capture agents combination 134 comprises two separate layers of different materials; one for molecular adhesion and one for capture agent. In some embodiments, the molecular adhesion layer and attachment of capture agents combination 134 comprises one single type of molecule or compound, that act as both the molecular adhesion layer and the attachment of capture agents. Namely, in some cases, the capture layer can have the property of the molecular adhesion layer, making the molecular adhesion unnecessary.
The nanostructure layer 132 and a molecular adhesion layer and attachment of capture agents combination 134 play many roles in detecting and amplifying to or from the analyte or both, or to or from a light signal by a light label attached to the analyte or both. These roles include but not limited to the following. The capture agent 134 selectively localize the targeted analytes while let other molecules and/or materials flow through.

The light amplification comes from one or several following factors: the nanosensor can (a) absorb light excitation effectively (e.g. the light at a wavelength that excites fluorescent moieties), (b) focus the absorbed light into certain locations, (c) place the analytes into the regions where most of light are focused, and (d) radiate efficiently the light generated by analytes from the locations where the analytes immobilized. The nanostructures also have the function to use their topology (shapes, sized, etc) localize and filters targeted analytes.

### Nanostructure Layer

With reference to FIGS. 1C, 1H and 1I, disclosed herein are several embodiments of nanostructure layer 132. One embodiment of nanostructure layer 132, termed “disk-on-complimentary-plane antenna” (DcP) 200 (FIG. 1D), comprises one or plurality of nanostructure element 290. The element 290 further comprises: a protrusion of dielectrics or semiconductors 220, extending from a surface of a wall of the fluidic channel; a metallic cap 230 on top of the protrusion 220; a metallic backplane 230 at the foot of the protrusion. The metallic backplane covers at least a portion of the fluidic channel wall surface near the foot of the protrusion; and the metallic cap 220 and the metallic backplane 250 are separated by a gap (i.e. separation) 215. The gap 215 does not need to be uniform. In some embodiments, a gap (i.e. separation) 215 is only at certain locations between the cap and backplane, while in some other locations of them the cap and backplane are in touch (i.e. zero gap). The backplane in each element has a hole surrounded the protrusion. The backplane 250 of each element 290 is connected to form a continuous metal film or substantially connected, or disconnected.

FIG. 1E shows another embodiment of nanostructure layer 132, termed “disc-coupled dots-on-pillar antenna arrays (D2PA)” 201, which is the same structure of DcP 200, except one or a plurality of nanodots 240 are on the sidewall of the protrusion 220. The nanostructure element 291 of D2PA 201 is also shown in FIG. 1E.

Another embodiment of nanostructure layer 132, shown in FIG. 1F, termed “Disc-on film antenna arrays (DoF)” 300, comprises one or plurality of nanostructure element 380, each of them further comprises a flat surface on a wall of the fluidic channel; a metallic flat and significantly continuous backplane 350 covers a portion of the flat surface; a protrusion of dielectrics or semiconductors 320 on top of the metallic backplane 350, occupying at least a portion of the metallic back plan surface; a metallic cap 330 on top of the protrusion; the metallic cap 330 and the metallic backplane 350 is separated by a gap (i.e. separation) 315. The gap 315 does not need to be uniform. In some embodiments, a gap (i.e. separation) 315 is only at certain locations between the cap and backplane, while in other locations of them he cap and backplane are in touch (i.e. zero gap). The backplane 350 of each element 390 is connected to form a continuous metal film or substantially connected, or disconnected.

Another embodiment of nanostructure layer 132, shown in FIG. 1G, termed “Disc-on-film-with-dots antenna arrays (DoFD)” 301, which is the same structure of DcP 300, except one or a plurality of nanodots 340 are on the sidewall of the protrusion 320. The nanostructure element 391 of DoFD 301 is also shown in FIG. 1G.

The examples of “disk-coupled dots-on-pillar antenna array, (D2PA)” and have been described (see, e.g., Li et al Optics Express 2011 19, 3925-3936 and WO2012/024006, which are incorporated by reference).

The arrange of the nanostructure elements in all embodiments can be either periodic or non-periodic depending upon the sensing performances, fabrication costs, and optical signals to be sensed. In general periodic structures are better than non-periodic for detection optical signal enhancement, but often hard or expensive to fabricate with a good precision.

Further, specifying parameters are described in WO2012/024006, which is incorporated by reference.

In some embodiments, the dimensions of one or more of the parts of the protrusions or a distance between two components may be that is less than the wavelength of the amplified light. For example, the lateral dimension of the protrusion body 220, the height of protrusion body 220, the dimensions of metal cap 230, the distances between any gaps between metallic dot structures 240, the distances between metallic dot structure 240 and metallic cap 230 may be smaller than the wavelength of the amplified light. As illustrated in FIG. 1A, the protrusions may be arranged on the substrate in the form of an array. In particular cases, the nearest protrusions of the array may be spaced by a distance that is less than the wavelength of the light. The protrusion array can be periodic and aperiodic.

### Examples of Nanostructures

The nanostructures in nanosensors use the similar material and dimensions. Here we use disc-coupled dots-on-pillar antenna array (D2PA) as examples, which should be applied to all nanostructures described herein. A disc-coupled dots-on-pillar antenna array (D2PA) has a 3D plasmon cavity antenna with a floating metallic cap or nanodisc that is coupled to nanoscale metallic dots on a protrusion body. Specifically, in some embodiments the D2PA has a substrate, a protrusion array on the substrate, a metallic cap or nanodisc on top of each of the protrusions, nanoscale metallic dots on the protrusion sidewall, with gaps between the cap and some of the dots, gaps between the neighboring dots, and a metallic back-plane which covers the most of the substrate areas that are not occupied by the protrusions. One difference in specification for the thickness of the metallic backplane: to consider plasmonic amplification of light signal, there is no restriction on the thickness for the examples of DcP 200 and DoF 300, but there is restriction for D2PA 201 and DoFD 301. The restrictions are given in discussions below.

A detailed description an exemplary D2PA that can be employed in a subject nanosensors are provided in WO2012/024006, which is incorporated by reference herein for disclosure for all purposes.

In one embodiment, the protrusion array is fabricated from SiO₂ with a 200 nm pitch, 130 nm height, and 70 nm diameter on the substrate, formed from silicon. The metallic back-plane may be formed from a 40 nm thick layer of gold, deposited on the protrusion array structures and substrate using e-beam evaporation along the normal direction. The deposition process forms the metallic caps in gold on top of each SiO₂ protrusion while simultaneously forming the gold nanohole metallic backplane on the surface of the silicon substrate. Each cap has a thickness of 40 nm and diameter about 110 nm. During the evaporation process, with a deposition rate of about 0.4 A/s, the gold atoms diffuse onto the sidewalls of the SiO₂ protrusions and congregate into random particles with granule sizes between 10 nm and 30 nm, forming the nanoscale metallic dots.

A substrate with the gold nanocaps, random gold nanoparticle metallic dots, and bottom gold nanohole plate (back-plane) is formed by the evaporation process. The gold nanoparticles scattered on the sidewall of the SiO₂ protrusions, forming the nanoscale metallic dots, have narrow gaps of about 0.5 nm-20 nm between them, which can induce highly enhanced electrical fields. As used herein, the term “gap” is defined as the minimum spacing between the two structures, such as the minimum spacing between two caps or the spacing between a cap and an adjacent dot structure. It also should be pointed out that the even a part of a dot contacts with another dot, an enhancement effects achieved by the present structures still exist, since there are other gaps present between adjacent structures in other locations.

The D2PA structure can enhance light absorption through plasma resonance and nanoantennas. The structure can enhance a local electric field through the nanogaps between the caps and nanodots and the nanogaps between the nanodots themselves, and assisted by the vertical cavity (for light) formed between the caps and the backplane, and the lateral cavity formed by the cap array.

More specifically, the structure can enhance the light absorption through the array of nanoprotrusions, and can enhance the reflection of an optical signal from the surface through these structures. It may have an enhanced vertical cavity light absorption effect, formed by the caps through the dots and the backplane to enhance the light absorption. It also can have a lateral cavity light absorption effect through the backplane of the metal to enhance the light absorption. It will be recognized by those skilled in the art that any particular D2PA structure may have one, several, or all of these functions, depending upon the specific configuration of the structure, including the spacing in the protrusion array, size of the protrusions, size of the caps, size of the dots, and materials employed.

The enhancement of optical signals by the structure will be a product of enhancement from the nanogaps between features of the structure, from plasmon resonance, from antenna absorptions, from antenna radiations, from vertical cavities, as well as lateral cavities. The elements and functions of D2PA structure may be viewed from a different angle. The caps and the holes in the backplane and the gap (i.e. spacing) between the cap and the adjacent metallic dots, as well as and between the dots themselves, can affect the local electric field enhancement provided by the structure. The dot position and number of dots on each protrusion body can also enhance the local electric field. The diameter of each protrusion and diameter of the capping cap can affect the plasmon resonant frequency. The silicon dioxide protrusion height can affect the cavity length and number of nanogaps, and also can affect the coupling of the cap and the gold backplanes. The number of protrusions per unit cell can affect the active areas, and the pitch (spacing) in the array of protrusions can affect coherent absorption and radiation of light. The gold backplane can affect the antenna and cavity, and the protrusion shape can determine the light dependent absorption.

Within the structure, multiple variables may be “tuned” to enhance signals. For example, the diameter of the caps and shape of the protrusions may be varied to alter the plasmon resonant frequency, the metallic dots will effect local signal enhancement, as well the cap-to-dot gap, dot position, and dot counts on each protrusion body; the height of the protrusions will affect the resonant cavity length and the number of nanogaps present, as well as the coupling effect between the cap and the metallic backplane. The total number of protrusions per unit cell on the surface of the structure defines the active areas, and the protrusion spacing (pitch) effects coherent absorption and radiation of optical energy. Finally, the metallic backplane material and thickness is related to antenna and cavity effects. Those of ordinary skill in this field will recognize that each of these variable may be altered as require from the exemplary embodiments shown herein to achieve a structure having desired characteristics or “tuning” to achieve specific enhancements, without departing from the scope of the present invention.

A variety of configurations for the structure are envisioned. For example, the structure of the D2PA can have a layer of SiO₂ under the metal backplane and which contiguously forms the protrusions. Alternatively, the D2PA having a metallic backplane without holes, such that the protrusions are formed directly on the backplane material, which in turn is deposited over a layer of SiO₂ on the underlying substrate.

When constructing the D2PA structure of the present disclosure, the material for the underlying substrate can be an insulator, a semiconductor, or a dielectric insulator. The substrate need not be monolithic, but may be of a laminate construction, comprising an insulator or semiconductor material top layer (the layer next to the protrusions) while the rest of the substrate is made of any solid material.

The protrusion bodies on the top layer of the substrate may be formed from an insulating material, but may be semiconductors. Exemplary materials for the formation of the protrusions are dielectrics: silicon-dioxide, silicon-nitride, hafnium oxide (HfO), Aluminum oxide (AlO) or semiconductors: silicon, GaAs, and GaN. Once formed, the protrusions may have sidewalls which are columnar (straight), sloped, curved, or any combination thereof. The height of each protrusion may be chosen from 5 nm to 7,000 nm, and a lateral dimension of each protrusion may be chosen from 5 nm to 8,000 nm. The shape of the top surface of the protrusion can be round, a point (of a pyramid), polygon, elliptical, elongated bar, polygon, other similar shapes or combinations thereof. The spacing between the protrusions in the array can be periodic or aperiodic. For some applications, a periodic period is preferred and the period is chosen to maximize the light absorption and radiation, which is light wavelength dependent. The spacing (pitch) between adjacent protrusions in the array may be from 4 nm to 4000 nm.

Each protrusion is topped with a metallic cap which may be formed from either: (a) single element metal, such as gold, silver, copper, aluminum, nickels; (b) a combination of the multiplayer and/or multilayer of the single metals; (c) metallic alloys; (d) semiconductors, (e) any other materials that generate plasmons, or (f) any combination of (a), (b), (c), (d) and (e). The shape of each cap can be a rounded, pointed (as in the form of a pyramid or cone), polygonal, elliptical, elongated bar, polygon, other similar shapes or combinations thereof. The shape of each cap can be the same as, or different from, the shape of the top surface of the associated protrusion on which it is disposed. Preferably, a lateral dimension of each cap is from 4 nm to 1500 nm, although in some embodiment 4 nm to 150 nm, and a thickness of the cap is from 1 nm to 500 nm, although in some embodiment 1 to 80 nm is preferred. The diameter of the metal caps can be either larger or smaller than the diameter of the supporting protrusion. The diameter difference can various from 0 to 200 nm depending the working wavelength.

Disposed on the sidewalls of each protrusion between the metallic cap and the metallic backplane, the metallic dots have shapes which are approximately spherical, discs-like, polygonal, elongated, other shapes or combinations thereof. The dimensions of the metallic dots are preferably between 1 nm to 200 nm, although in some embodiment, 1 nm to 100 nm are preferred, and may be different in three dimensions. The exact dimension of the dots may be selected for a specific light signal, as well regulated by fabrication convenience and the fabrication of the associated gaps there between.

In some embodiments, the gaps between the neighboring metallic dots and the gap between the cap and adjacent metallic dots is between 0.1 nm to 200 nm, although the preferred range is 0.1 nm to 60 nm. For many applications, a small gap is preferred to enhance the optical signals. The gaps may be varied between each metallic dot on a protrusion.

In the embodiment, the metallic backplane defines a metallic layer on the substrate with a hole for each protrusion. The thickness of the metallic backplane is selected to be from 1 nm to 2000 nm, with a thickness in the range of 50 nm-200 nm preferred. The material of the metallic backplane can be selected from the same group as is used to form the metallic cap described above, but for a given D2PA structure, the metallic backplane can be formed from either the same or a different material as that used to form the caps.

The above descriptions of the D2PA structure are illustrative of the range of the materials, shapes, and dimensions which may be employed, but are not considered to be exclusive. Other materials, shapes, and dimensions may be used as required to achieve a desired enhancement effect. The exact materials, shapes, and dimensions for each D2PA structure will be determined by particular requirements imposed by the light absorption to be enhanced (wavelength, polarization), the light re-radiation to be enhanced, and/or the local electric field to be enhanced.

A D2PA array may be fabricated using the following method. The initial step is to provide the substrate with a layer of protrusion material, such as SiO₂. The next step is to employ a lithographic imprinting process to imprint a mold having a pattern of protrusions into a resist layer deposited over the layer of protrusion material. After imprinting the pattern into the resist layer to create an etch mask, the residual material is removed via an etching process to leave a pattern of protrusion-like structures of the resist layer. A layer of etch mask material, such as chromium (Cr) or other material is then deposited over the pattern of protrusion-like structures, and the remaining resist material removed, resulting in a pattern of Cr deposited directly on the layer of protrusion material. A final etching step which may be a dry etching such as retro-etching, or a wet etching process, removes the unprotected portions of protrusion material, and leaves an array of protrusions disposed on the surface of the substrate. Any remaining etch mask material (Cr) is optionally removed by either a dry or wet etching process, and an evaporation process is employed to deposit the metallic backplane material, cap material, and metallic dots onto the structure in a substantially collimated deposition.

Those of ordinary skill will recognize that the various lithography steps can use any variety of known lithography methods, including electron-beam lithography, ion beam lithography, photolithography, or nanoimprint lithography to form the pattern in the resist material. Similarly, it will be recognized that the etching mask material can be metal dielectric or insulators.

The etch mask material can be deposited on the resist layer before or after the lithography step is performed. A liftoff process will typically be used if the etch masking material is deposited after the lithography step. Alternatively, if the step of nanoimprint lithography is used to create a resist pattern first, an etch mask material may be subsequently deposited into the resulting trenches second, and then a liftoff process is performed. Other methods for making a D2PA array are possible.

Through manipulation of the various parameters of the D2PA structure, light of various wavelengths from about 100 nm to about 8000 nm may be manipulated.

The enhancement structure may be constructed with one or more features specific to the wavelength of light to be detected. These features include including the material selection, the nanoscale protrusion height, the nanoscale protrusion sidewall shape, the nanoscale metallic cap shape, the nanoscale metallic dot structure spacing, the metallic materials, and the metallic backplane configuration. The selection of the nanoscale metallic dot structure spacing further includes selecting a gap distance between adjacent nanoscale metallic dot structures and/or selecting a gap spacing between the nanoscale metallic cap and adjacent nanoscale metallic dot structures.

The substrate of the nanoscale structure may be an electrical insulator, a dielectric insulator or a semiconductor. Optionally the substrate may be a laminate structure, and wherein a layer at the surface of the substrate is either an electrical insulator or a semiconductor; and wherein a body of the substrate below the surface layer consists of any solid material.

The protrusion bodies may be formed from either an insulator or a semiconductor, and has a top which has a shape selected from the group of shapes consisting of round, pointed, polygonal, pyramidal, elliptical, elongated bar shaped, or any combinations thereof. The sidewall surface of the protrusion may be columnar, sloped, or curved. Preferably, the protrusion has a height in the range from 5 nm to 7000 nm and a diameter in the range from 5 nm to 8000 nm. Optionally, the protrusion may be part of an array of protrusions extending from the surface of the substrate, with a spacing between adjacent protrusions in the range from 2 nm to 4,000 nm. The array of protrusions may define a periodic array with a spacing selected in relation to light of a selected wavelength in order to maximize absorption or radiation of the light using the nanoscale structure. Suitable materials for the formation of the protrusions on the nanoscale structure include silicon-dioxide, silicon-nitride, hafnium oxide, aluminum oxide, silicon, gallium arsenide, and gallium nitride.

The metallic caps of the nanoscale structure are formed on top of the protrusions from a metal such as gold, silver, copper, aluminum, alloys thereof, or combinations thereof. The surface of the metallic caps need not be uniform, and may have any configuration such as round, pointed, polygonal, elliptical, bar or combinations thereof. Preferably, a lateral dimension of the metallic cap is in the range from 5 nm to 1500 nm and a vertical thickness of the metallic cap is in the range from 1 nm to 500 nm.

The metallic dot structures disposed on the protrusion sidewalls of the nanoscale structure each have a shape selected from a group of shapes consisting of approximately spherical, circular, polygonal, elongated or combinations thereof, and have dimensions in the range 1 nm to 600 nm, although in some embodiment, 1 to 60 nm are preferred. A gap between the metallic dot structures and the metallic cap on a common protrusion is in a range from 0.5 nm to 600 nm, as is the gap between adjacent metallic dot structures.

The metallic backplane of the nanoscale structure may be configured either with holes through which the protrusion bodies extend from surface of the substrate, or may be substantially continuous, with the protrusion bodies disposed there on. Preferably, the metallic backplane has a thickness ranging from 1 nm to 2000 nm, e.g., from 50 nm to 200 nm, and is composed of a metal selected from the group of metals consisting of gold, silver, copper, aluminum, alloys thereof, or combinations thereof. The metallic backplane may be formed from either the same material as, or a different material from, the metallic caps.

The nanoscale structure of the present disclosure may be made by a variety of methods. An exemplary method for manufacture of the nanoscale structure for enhancing local electric fields, absorbing light or radiating light comprises the steps of: providing a substrate comprising an outer surface of insulating or semiconductive material; forming on the outer surface an array of protrusions having a height in the range 5 nm to 7000 nm and a lateral dimension in the range 5 nm to 8000 nm; applying conductive material to the tops of the protrusions and to the underlying substrate; and simultaneously (or subsequently) depositing conductive dot structures on the protrusion sidewalls. The array of protrusions is formed by a process comprising electron beam lithography, ion-beam lithography, photolithography or nanoimprint lithography.

In one embodiment that is configured for enhance light at a wavelength of ˜800 nm, the D2PA nanostructure may be composed of a periodic non-metallic (e.g. dielectric or semiconductor) protrusion array (200 nm pitch and ˜100 nm diameter), a metallic disk (˜135 nm diameter) on top of each protrusion, a metallic backplane on the foot of the protrusions, metallic nanodots randomly located on the protrusion walls, and nanogaps between these metal components. The disk array and the backplane (both are 55 nm thick) form a 3D cavity antenna that can efficiently traps the excitation light vertically and laterally. Each protrusion has about 10 to 50 nanodots depending upon the protrusion geometry; and the protrusion density is 2.5×10⁹ protrusions/cm².

The device may be configured to detect light having a wavelength in the range of 400 to 1,000 nm range. In certain embodiments, the average diameter for the nanodots is in the range of 1 nm to 25 nm, and gaps between the nanodots, and the gaps between the nanodots and the nanodisks may be in the range of 1 nm to 10 nm. The metal is selected from the group consisting of gold, silver, copper, aluminum, alloys thereof, and combinations thereof. The top of the protrusion has a shape selected from the group of shapes consisting of round, polygonal, pyramidal, elliptical, elongated bar shaped, or any combination thereof. The lateral dimension of the metallic cap is in the range from 5 nm to 150 nm. The metallic cap and the metallic backplane are spaced by a distance in the range of 0.1 nm to 60 nm. At least one metallic dot structure has dimensions in the range of 1 nm to 25 nm. The distance between the metallic dot structure the metallic cap, and the distance between the metallic dot structure and the metallic backplane are spaced by a distance in the range of 0.5 nm to 50 nm.

In particular embodiments, the spacing between the two nearest protrusions of the plurality of protrusions is in the range from 2 nm to 200 nm. The protrusion has a sidewall surface that is columnar, sloped, or curved. The thickness of the metallic cap and metallic backplane is between 5 nm to 60 nm. The protrusion has a lateral dimension or a height less than the wavelength of the light. The metallic cap has substantially the same lateral geometry as the protrusion. The protrusion comprises a dielectric or semiconductor material selected from the group consisting of polymers, silicon-dioxide, silicon-nitride, hafnium oxide, aluminum oxide, silicon, gallium arsenide, and gallium nitride. The lateral dimension of the metallic cap is less than the wavelength of the light.

### Molecular Adhesion Layer and Attachment of Capture Agents

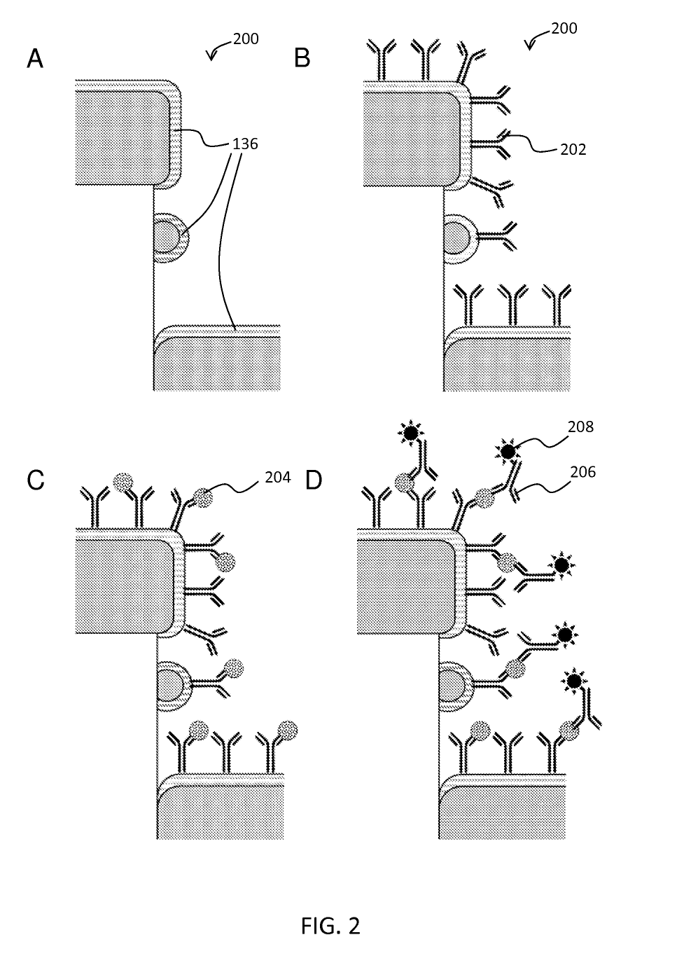

With reference to FIGS. 2 to 5, the molecular adhesion layer and attachment of capture agents combination 134 comprises, in some embodiment, two separate layers of different materials: molecular adhesion layer 136 and capture agent attached; and in some other embodiment, one single type of molecule or compound, that act as both the molecular adhesion layer and the attachment of capture agents. Namely, in some cases, the capture layer can have the property of the molecular adhesion layer, making the molecular adhesion unnecessary. The molecular adhesion layer 136 may cover only a portion of the nanostructure 132 and may be elective to the materials in the nanostructure 132 (e.g. only the metal of the nanostructure layer). And the capture agent may attached to a portion of The molecular adhesion layer 136.

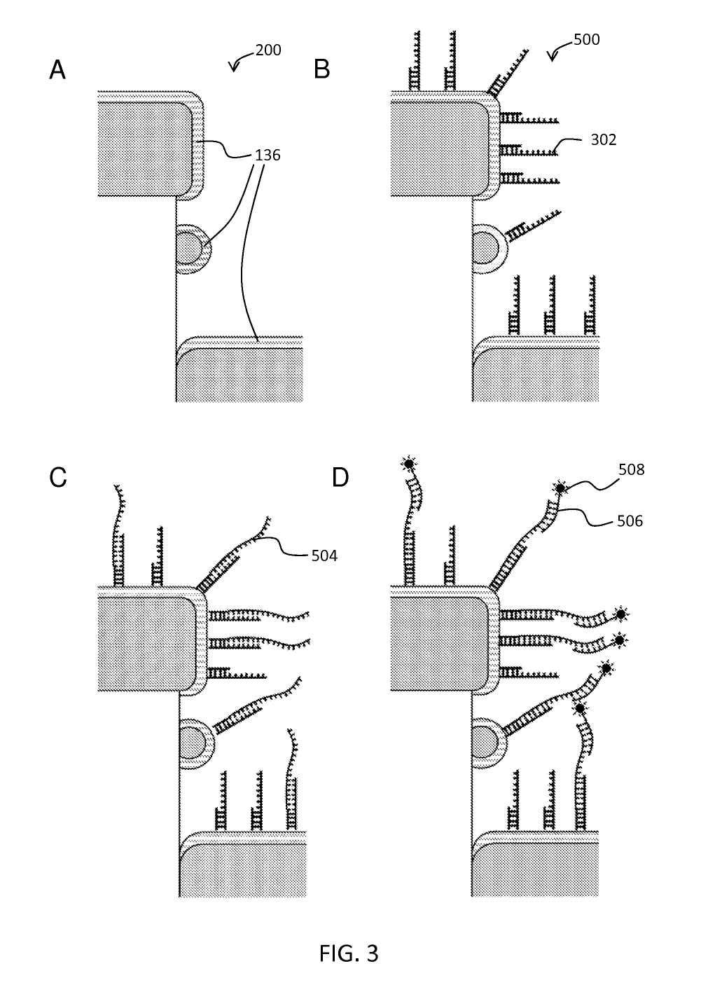

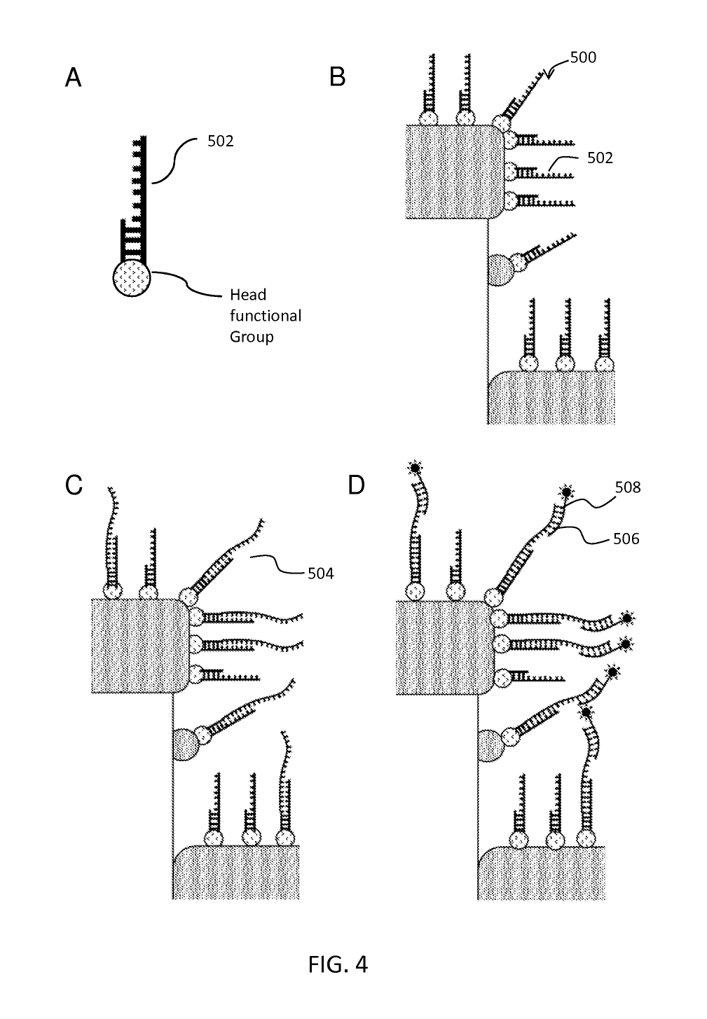

With reference to FIGS. 2, and 3, examples of the capture agent are antibody 202 or nucleic acid 500. The capture agent also can be antigen. With reference to FIG. 4, the nucleic acid molecular 502 has a functional group that can directly attach to the metal of the nanostructure 132 without additional molecular adhesion layer, therefore serving the function of both the molecular adhesion layer and the capture agent.

As shown in FIG. 1I is a process how of applying an antibody capture agent 202 on the molecular adhesion layer 136, capture a targeted analyte 204, and label the analyte using an detection agent 208.

The a molecular adhesion layer 136 covers at least a part of the metal surfaces of the underlying D2PA. The molecular adhesion layer has two purposes. First, the molecular adhesion layer acts a spacer. For optimal fluorescence, the light-emitting labels (e.g., fluorophores) cannot be too close to the metal surface because non-radiation processes would quench fluorescence. Nor can the light-emitting labels be too far from the metal surface because it would reduce amplification. Ideally, the light-emitting labels should be at an optimum distance from the metal surface. Second, the molecular adhesion layer provides a good adhesion to attach capture agent onto the nanosensors. The good adhesion is achieved by having reactive groups in the molecules of the molecular adhesion layer, which have a high affinity to the capture agent on one side and to the nanosensors on the other side.

With reference to FIGS. 2 and 3, the exterior surface of molecular adhesion layer 136 comprises a capture-agent-reactive group, i.e., a reactive group that can chemically react with capture agents, e.g., an amine-reactive group, a thiol-reactive group, a hydroxyl-reactive group, an imidazolyl-reactive group and a guanidinyl-reactive group. For illustrative purposes, the molecular adhesion layer 136 covers all of the exposed surface of metallic dot structure 250, metal cap 230, and metallic backplane 250. However, in some embodiments, adhesion layer 136 need only part of the exposed surface of metallic dot structure 250, metal cap 230, or metallic backplane 250. As shown, in certain cases, substrate 110 may be made of a dielectric (e.g., SiO₂) although other materials may be used, e.g., silicon, GaAs, polydimethylsiloxane (PDMS), poly(methyl methacrylate) (PMMA). Likewise, the metal may be gold, silver, platinum, palladium, lead, iron, titanium, nickel, copper, aluminum, alloy thereof, or combinations thereof, although other materials may be used, as long as the materials' plasma frequency is higher than that of the light signal and the light that is used to generate the light signal.

The molecular adhesion layer (MAL) 136 can have many different configurations, including (a) a self-assembled monolayer (SAM) of cross-link molecules, (b) a multi-molecular layers thin film, (c) a combination of (a) and (b), and (d) a capture agent itself.

In the embodiment of MAL (a), where the molecular adhesion layer 136 is a self-assembled monolayer (SAM) of cross-link molecules or ligands, each molecule for the SAM comprises of three parts: (i) head group, which has a specific chemical affinity to the nanosensors's surface, (ii) terminal group, which has a specific affinity to the capture agent, and (iii) molecule chain, which is a long series of molecules that link the head group and terminal group, and its length (which determines the average spacing between the metal to the capture agent) can affect the light amplification of the nanosensors. Such a SAM is illustrated in FIG. 3.

In many embodiments, the head group attached to the metal surface belongs to the thiol group, e.t., —SH. Other alternatives for head groups that attach to metal surface are, carboxylic acid (—COOH), amine (C═N), selenol (—SeH), or phosphane (—P). Other head groups, e.g. silane (—SiO), can be used if a monolayer is to be coated on dielectric materials or semiconductors, e.g., silicon.

In many embodiments, the terminal groups can comprise a variety of capture agent-reactive groups, including, but not limited to, N-hydroxysuccinimidyl ester, sulfo-N-hydroxysuccinimidyl ester, a halo-substituted phenol ester, pentafluorophenol ester, a nitro-substituted phenol ester, an anhydride, isocyanate, isothiocyanate, an imidoester, maleimide, iodoacetyl, hydrazide, an aldehyde, or an epoxide. Other suitable groups are known in the art and may be described in, e.g., Hermanson, “Bioconjugate Techniques” Academic Press, 2nd Ed., 2008. The terminal groups can be chemically attached to the molecule chain after they are assembled to the nanosensors surface, or synthesized together with the molecule chain before they are assembled on the surface.

Other terminal groups are Carboxyl —COOH groups (activated with EDC/NHS to form covalent binding with —NH2 on the ligand); Amine, —NH2, group (forming covalent binding with —COOH on the ligand via amide bond activated by EDC/NHS); Epoxy, Reacted with the —NH2 (the ligand without the need of a cross-linker); Aldehyde, (Reacted with the —NH2 on the ligand without the need of a cross-linker); Thiol, —SH, (link to —NH2 on the ligand through SMCC-like bioconjugation approach); and Glutathione, (GHS) (Ideal for capture of the GST-tagged proteins.

The molecular chain can be carbon chains, their lengths can be adjusted to change the distance between the light emitting label to the metal for optimizing the optical signal. In one embodiment, as will be described in greater detail in example section, the SAM layer is dithiobis(succinimidyl undecanoate), whose head group is —SH that binds to gold surface through sulfer-gold bond, and terminal group is NHS-ester that bind to the primary amine sites of the capture agent, and the molecule alkane chain with length of 1.7 nm.

In many embodiments, the molecule chains that link head groups and terminal groups are alkane chain, which is composed of only hydrogen and carbon atoms, with all bonds are single bonds, and the carbon atoms are not joined in cyclic structures but instead form a simple linear chain. Other alternatives for molecule chain can be ligands that are from polymers such as poly(ethylene glycol) (PEG), Poly(lactic acid) (PLA), etc. The molecule chains are chemically non-reactive to neither the metal surface that the head groups attach to, nor the capture agent that the terminal groups attach to. The chain length, which determines the distance of analyte to the nanosensors's surface, can be optimized in order to achieve the maximum signal amplification. As will be described in greater detail below, the molecule chains may have a thickness of, e.g., 0.5 nm to 50 nm.

The molecular adhesion layer used in the subject nanosensor may be composed of a self-assembled monolayer (SAM) that is strongly attached to the metal at one side (via, e.g., a sulfur atom) and that terminates a capture-agent-reactive group, e.g., an amine-reactive group, a thiol-reactive group, a hydroxyl-reactive group, an imidazolyl-reactive group and a guanidinyl-reactive group, at the other (exterior) side. The monolayer may have a hydrophobic or hydrophilic surface. The most commonly used capture-agent reactive groups are NHS (which is amine-reactive) and maleimide (which is sulfhydrl-reactive), although many others may be used.

In some embodiments, the molecular adhesion layer may be a self-assembled monolayer of an alkanethiol (see, e.g., Kato Journal of Physical Chemistry 2002 106: 9655-9658), poly(ethylene)glycol thiol (see, e.g., Shenoy et al Int. J. Nanomedicine. 2006 1: 51-57), an aromatic thiol or some other chain that terminates in the thiol.

Thiol groups may be used because (a) the thiol sulfur interacts with gold and other metals to form a bond that is both strong and stable bond (see, e.g., Nuzzo et al J. Am. Chem. Soc. 1987 109:2358-2368) and (b) van der Waals forces cause the alkane and other chains chains to stack, which causes a SAM to organize spontaneously (see, e.g., Love et al. Chem. Rev. 2006 105:1103-1169). Further, the terminal group is available for either direct attachment to the capture molecule or for further chemical modifications.

Alkanethiol may be used in some embodiments. It has been estimated that there are 4×10¹⁴ alkanethiol molecules/cm² in a packed monolayer of alkanethiol (Nuzzo et al, J. Am. Chem. Soc. 1987 109:733-740), which approximately corresponds to an alkanethiol bond to every gold atom on the underlying surface. Self-assembled monolayers composed of alkanethiol can be generated by soaking the gold substrate in an alkanethiol solution (see, e.g., Lee et al Anal. Chem. 2006 78: 6504-6510). Gold is capable of reacting with both reduced alkanethiols (—SH groups) and alkyldisulfides (—S—S—) (see, e.g., Love et al Chem. Rev. 2005 105:1103-1169).

Once a self-assembled monolayer of poly(ethylene)glycol thiol or alkanethiol has been produced, a large number of strategies can be employed to link a capture to the self-assembled monolayer. In one embodiment, a capture agent such as streptavidin (SA) can be attached to the SAM to immobilize biotinylated capture agents.

In one embodiment, streptavidin (SA) itself can be use as a functional group (e.g. terminal group) the SAM to crosslink capture agent molecules that have high binding affinity to SA, such as biotinylated molecules, including peptides, oligonucleotides, proteins and sugars.

The functional group of avidin, streptavidin have a high affinity to the biotin group to form avidin-biotin. Such high affinity makes avidin/streptavidin serve well as a functional group and the biotin group as complementray functional group binding. Such functional group can be in binding the molecular adhesion layer to the nanosensors, in binding between molecular adhesion layer and the capature agent, and in binding a light emitting lable to the secondary capture agent. In one embodiment, a molecular adhesion layer containing thiol-reactive groups may be made by linking a gold surface to an amine-terminated SAM, and further modifying the amine groups using sulfosuccinimidyl-4-(N-maleimidomethyl)cyclohexane-1-carboxylate (Sulfo-SMCC) to yield a maleimide-activated surface. Maleimide-activated surfaces are reactive thiol groups and can be used to link to capture agents that contain thiol- (e.g., cysteine) groups.

In another embodiment, a molecular adhesion layer containing an amine-reactive group (N-hydroxl succinimide (NHS)) can be produced by, e.g., by soaking the gold substrate in a 1-10 mM solution of succinimidyl alkanedisulfides such as dithiobis-sulfosuccinimidylpropionate (DSP) or dithiobis(succinimidyl undecanoate) (see, e.g., Peelen et al J. Proteome Res. 2006 5:1580-1585 and Storri et al Biosens. Bioelectron. 1998 13: 347-357).

In another embodiment, a molecular adhesion layer containing an amine-reactive group (NHS) may be produced using carboxyl-terminated SAM such as 12-carboxy-1-undecanethiol. In this case, the surface of the SAM may be linked to the NHS in the presence of 1-ethyl-3(3dimethylaminopropyl)carbodiimide HCl (EDC) to yield an inter-mediate which forms stable amide bonds with primary amines (see, e.g., Johnsson et al Anal. Biochem. 1001 198: 268-277).

In another embodiment, a molecular adhesion layer may contain Protein A which binds with high affinity to Fc region of IgGs, other immunoglobulin form, e.g., IgE.

In another embodiment, an imidazole group (which is also reactive with amines) may be added by reacting a carboxyl-terminated SAM with 1,1′-carbonyldiimidazole (CDI).

In further embodiments, aldehyde-terminated alkanethiol monolayers can be used to immobilize both proteins and amine-terminated DNA oligonucleotides, and his-tagged fusion proteins can be immobilized on nitrilotriacetic (NTA)-modified gold surfaces.

Thiol-reactive groups can link to synthetic DNA and RNA oligonucleotides, including aptamers, which can be readily synthesized commercially with a thiol terminus. Thiol-reactive groups can also link to proteins that contain a cysteine groups, e.g., antibodies. Thiolated molecules can be attached to maleimide-modified surfaces (see, e.g., Smith et al Langmuir 2002 19: 1486-1492). For in certain cases, one may use an amino acid spacer (e.g., Ser-Gly-Ser-Gly) inserted after a terminal Cys, which improves the amount of binding relative peptides that lacking spacers. For oligonucleotides, an alkane spacer can be used. Carbohydrates synthesized to contain with terminal thiols can be been tethered to gold in the same way.

Amine-reactive groups can form bonds with primary amines, such as the free amine on lysine residues. In addition to proteins, amine-reactive surfaces can be used to immobilize other biomolecules, including peptides containing lysine residues and oligonucleotides synthesized with an amine terminus.

In the embodiment of MAL (b), in which the molecular adhesion layer 136 is a multi-molecular layer thin film, the molecules may be coated on the D2PA nanosensors through physical adsorption or strong binding. In one example, protein A can be coated over the entire or partial areas of the surface of D2PA nanosensors surface, in which case the protein A can be deposited through physical adsorption process and has a thickness of 4 nm to 5 nm. In another example, the layer may be a thin film of a polymer such as polyethylene glycol (PEG), which has a functional head group on one end, e.g., thiol (—SH). The functioned PEG molecule layer forms a strong bond to D2PA nanosensors's surface. The thickness of PEG molecule layer can be tuned by changing the PEG polymer chain length. Another example is an amorphous SiO2 thin film, which is attached to the surface of the D2PA nanosensors using physical or chemical deposition methods, e.g., evaporation, sputtering, sol-gel method. The thickness of the SiO2 thin film can be precisely controlled during the deposition.

In the embodiment of MAL (c), where the molecular adhesion layer 136 is a combination of a multi-molecular layer thin film and a SAM, the SAM layer may be deposited first, followed by a multi-molecular layer.

In one example, the molecular adhesion layer may contain a monolayer of streptavidin first, followed by other layers of molecules that have high binding affinity to streptavidin, such as biotin, biotinylated molecules, including peptides, oligonucleotides, proteins, and surgars.

In one example, the molecular adhesion layer, may contain a SAM layer dithiobis(succinimidyl undecanoate) (DSU) and a Protein A layer. The DSU SAM layer binds to nanosensors's metal surface through sulfer-gold bond, and has a terminal group of NHS-ester that binds to the primary amine sites on Protein A. In a particular case, capture antibodies bond to such bilayer of protein A on top of DSU through their Fc region. The protein A can ensure the orientation of antibodies for better capture efficiency.

In the embodiment of MAL (d), where the molecular adhesion layer 136 is a capture agent itself, the capture agent has a headgroup that have a high affinity to the metal or protrusion sidewall of the subject nanosensors (i.e. D2PA). One of the common headgroup is thiol-reactive group. Thiol-reactive groups can link to synthetic DNA and RNA oligonucleotides, including aptamers, which can be readily synthesized commercially with a thiol terminus. Thiol-reactive groups can also link to proteins that contain a cysteine groups, e.g., antibodies. Another example where the MAL itself is used as the capture agent is a layer of antibody fragments, e.g., half-IgG, Fab, F(ab′)2, Fc. The antibody fragments bond to metal surface directly through the thiol-endopeptidase located in the hinge region. This embodiment is illustrated in FIG. 4. In this embodiment, the nucleic acid comprises a headgroup that binds directly the nanosensors. The remainder of the steps are performed as described.

The thickness of molecular adhesion layer should be in the range of 0.5 nm to 50 nm, e.g., 1 nm to 20 nm. The thickness of the molecular adhesion layer can be optimized to the particular application by, e.g., increasing or decreasing the length of the linker (the alkane or poly(ethylene glycol) chain) of the SAM used. Assuming each bond in the linker is 0.1 nM to 0.15 nM, then an optimal SAM may contain a polymeric linker of 5 to 50 carbon atoms, e.g., 10 to 20 carbon atoms in certain cases.

A nanosensor may be made by attaching capture agents to the molecular adhesion layer via a reaction between the capture agent and a capture-agent reactive group on the surface of the molecular adhesion layer.

Capture agents can be attached to the molecular adhesion layer via any convenient method such as those discussed above. In many cases, a capture agent may be attached to the molecular adhesion layer via a high-affinity strong interactions such as those between biotin and streptavidin. Because streptavidin is a protein, streptavidin can be linked to the surface of the molecular adhesion layer using any of the amine-reactive methods described above. Biotinylated capture agents can be immobilized by spotting them onto the streptavidin. In other embodiments, a capture agent can be attached to the molecular adhesion layer via a reaction that forms a stong bond, e.g., a reaction between an amine group in a lysine residue of a protein or an aminated oligonucleotide with an NHS ester to produce an amide bond between the capture agent and the molecular adhesion layer. In other embodiment, a capture agent can be strongly attached to the molecular adhesion layer via a reaction between a sulfhydryl group in a cysteine residue of a protein or a sulfhydrl-oligonucleotide with a sulfhydryl-reactive maleimide on the surface of the molecular adhesion layer. Protocols for linking capture agents to various reactive groups are well known in the art.

In one embodiment, capture agent can be nucleic acid to capture proteins, or capture agent can be proteins that capture nucleic acid, e.g., DNA, RNA. Nucleic acid can bind to proteins through sequence-specific (tight) or non-sequence specific (loose) bond.

In certain instances, a subject nanosensors may be fabricated using the method: (a) patterning at least one protrusion on a top surface of a substrate; (b) depositing a metallic material layer of the top surface; (c) allowing the metallic material deposited on the protrusion tops to form a cap, the metallic material deposited on the protrusion feet to form a metallic backplane, and the metallic material deposited on the sidewall to form at least one metallic dot structure; and, as described above, (d) depositing a molecular adhesion layer on top of the deposited metallic material, wherein the molecular adhesion layer covers at least a part of the metallic dot structure, the metal cap, and/or the metallic backplane, and wherein the exterior surface of the molecular adhesion layer comprises a capture agent-reactive group.

Furthermore, the patterning in (a) include a direct imprinting (embossing) of a material, which can be dielectric or semiconductor in electric property, and can be polymers or polymers formed by curing of monomers or oligomers, or amorphous inorganic materials. The material can be a thin film with a thickness from 10 nanometer to 10 millimeter, or multilayer materials with a substrate. The imprinting (i.e. embossing) means to have mold with a structure on its surface, and press the mold into the material to be imprinted to for an inverse of the structure in the material. The substrates or the top imprinted layers can be a plastic (i.e. polymers), e.g. polystyring (PS), Poly(methyl methacrylate) (PMMA), Polyethylene terephthalate (PET), other acrylics, and alike. The imprinting may be done by roll to roll technology using a roller imprinter. Such process has a great economic advantage and hence lowering the cost.

### Fabrication of Microfluidic Devices

The microfluidic devices 100 can be fabricated in various ways. Some of examples are given herein. One method in fabricating the key components of the microfluidic device 100 comprises (a) patterning at least one protrusion on a surface of a substrate, the protrusion occupies, after the patterning, a portion of the surface; (b) depositing a metallic material layer to the top of the protrusion and an area of the surface that is not occupied by the protraction, where in the depositions occur in parallel; (c) patterning a microfluidic channel around the protrusion, wherein the patterning is before or after, or partially before and partially after the protrusion patterning and the metal deposition; wherein the protrusion and the metallic structures form the nanostructures of the microfluidic device.

The fabrication method above, wherein the deposition of metallic material further comprises depositing the same metallic material on protrusion sidewall in the same process as the deposition on the protrusion top and the open area of the surface, the same metallic material on sidewall of the protrusion. The method of fabrication of claims 4, wherein the method of fabrication further comprises depositing a metallic layer on the surface before the patterning of the protrusion.

Another method in fabricating the key components of the microfluidic device 100 comprises (a) depositing and patterning a lift-off template layer on a surface of a substrate, the lift-off layer has a hole that exposing the substrate surface; (b) depositing materials needed for the metallic structures and dielectric/semiconductor protrusion from the top of lift-off template, a portion of the deposited material is inside the hole and in contact with the substrate surface and a portion of the deposited materials is on top surface of the lift-off template and not directly in contact with the substrate surface; (c) dissolving the lift-off template in a solution, wherein the materials deposited on the top of lift-off template is separated from the substrate and the materials deposited inside the hole is remain on the substrate, (d) patterning a microfluidic channel around the protrusion, wherein the patterning is before or after, or partially before and partially after the dissolving of the lift-off template.

In both fabrication methods above method of fabrication, the method of patterning comprises nanoimprint, and the method of fabrication further comprises depositing a capture agent for sensing an analyte onto a nanosensor in the microfluidic device, wherein the deposition is either before or after the patterning of the microfluidic channel. Furthermore, in the fabrication above, the patterning a microfluidic channel around the protrusion after the protrusion patterning and the metal deposition comprises (i) fabricating open microfluidic channels on anther substrate, (ii) bonding the substrate with a substrate with the protrusion and the metallic materials; wherein the substrates are aligned and the protrusion is inside a microfluidic channel after the bonding.

### Systems

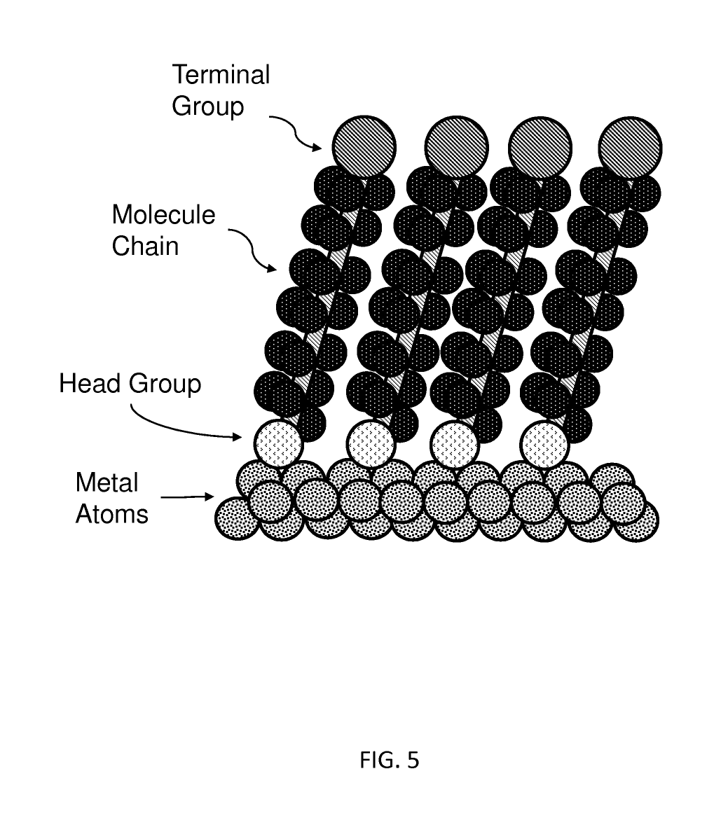

With reference to FIG. 5, also provided is a system 400 comprising a subject nanosensor, a holder for the nanosensor (not shown), an excitation source 410 that induces a light signal from a label (i.e. light emitting label); and a reader 420 (e.g., a photodetector, a CCD camera, a CMOS camera, a spectrometer or an imaging device capable of producing a two dimensional spectral map of a surface of the nanosensor) adapted to read the light signal. As would be apparent, the system may also has electronics, computer system, software, and other hardware that amplify, filter, regulate, control and store the electrical signals from the reader, and control the reader and sample holder positions. The sample holder position can be move in one or all three orthogonal directions to allow the reader to scan the light signal from different locations of the sample.

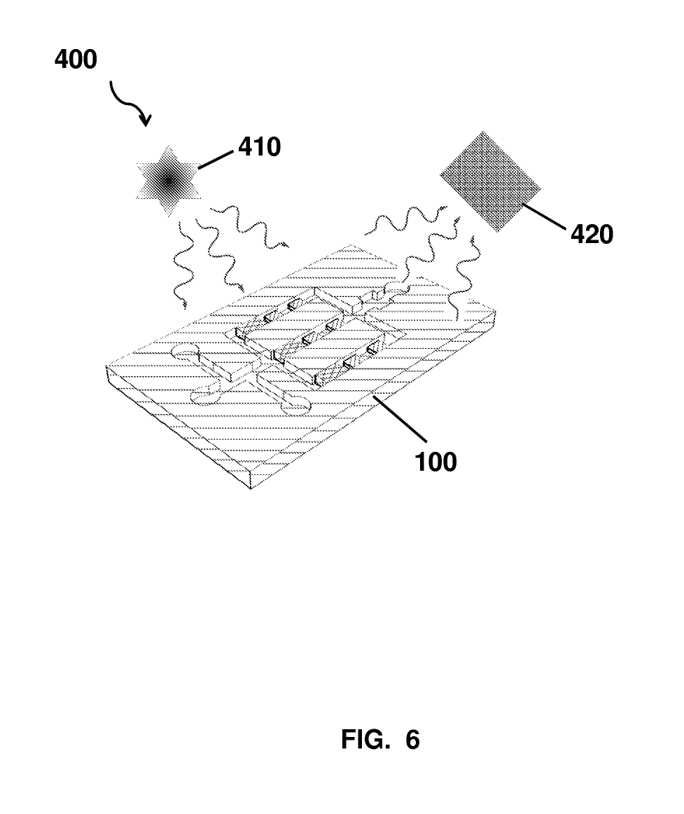

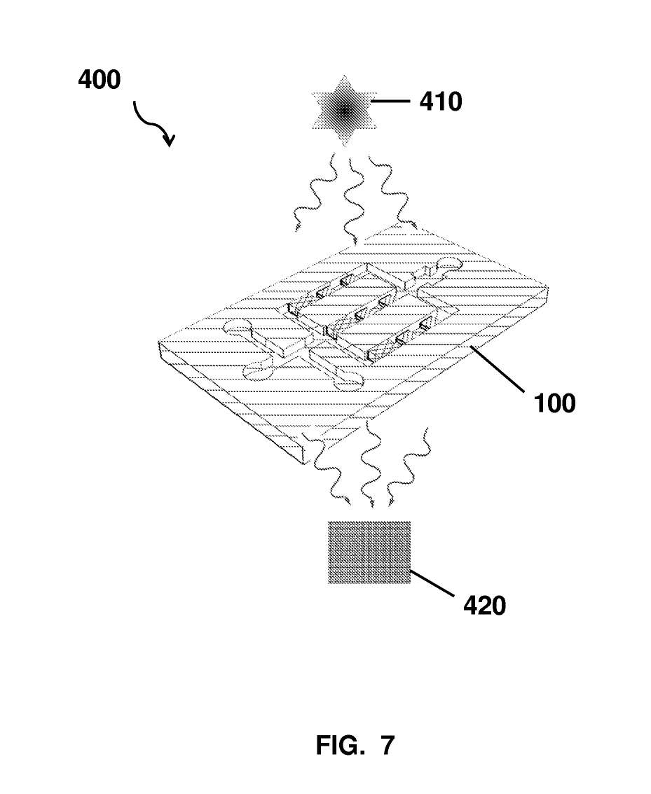

The excitation source may be (a) a light source, e.g., a laser of a wavelength suitable for exciting a particular fluorophore, and a lamp or a light emitting diode with a light filter for wavelength selection; or (b) a power source for providing an electrical current to excite light out of the nanosensor (which may be employed when an electrochemiluminescent label is used). An exemplary system is illustrated in FIGS. 6 and 7. With reference to FIGS. 6 and 7, the excitation system may comprise a laser, laser optics (including a beam expander, lens, mirror and a laser line-pass filter), a reader (e.g., a spectrometer with a CCD sensor), further optics (e.g., a long wavelength pass filter, a beam splitter, and a lens), and a holder for the nanosensor. In certain cases, the holder may be on a motorized stage that has an X-Y and Z movement.

In particular cases, laser-line pass filter filters out light whose wavelength is different from the laser, and the long wavelength pass filter will only allow the light emanate from the optically detectable label to pass through. Since different fluorescence labels absorb light in different spectral range, the fluorescence label should be chosen to match its peak absorption wavelength to the laser excitation wavelength in order to achieve optimum quantum efficiency. In many embodiments, the light signal emanating from the fluorescence label on the nanosensors are at a wavelength of at least 20 nm higher than the laser wavelength. Thus the nanosensor's plasmonic resonance should be tuned to cover the fluorescence label's absorption peak, emission peak and laser excitation wavelength. In some embodiments, the excitation and fluorescence wavelength range can be from 100 nm to 20,000 nm. The preferred range is from 300 nm to 1200 nm. The 600-850 nm range is preferable due to low background noise.

The system, wherein the excitation source is from a group of light sources (laser and light emitting diode), electrical source (e.g. power supplies), and chemical source (e.g. chemicals).

The system, wherein the reader is selected from a photodetector, a CCD camera, a CMOS camera, a spectrometer or an optical sensor, that is capable of producing a zero, one, two, or three dimensional information of the property of light from the nanosensor.

System Integrated with Mobile Smart Phone

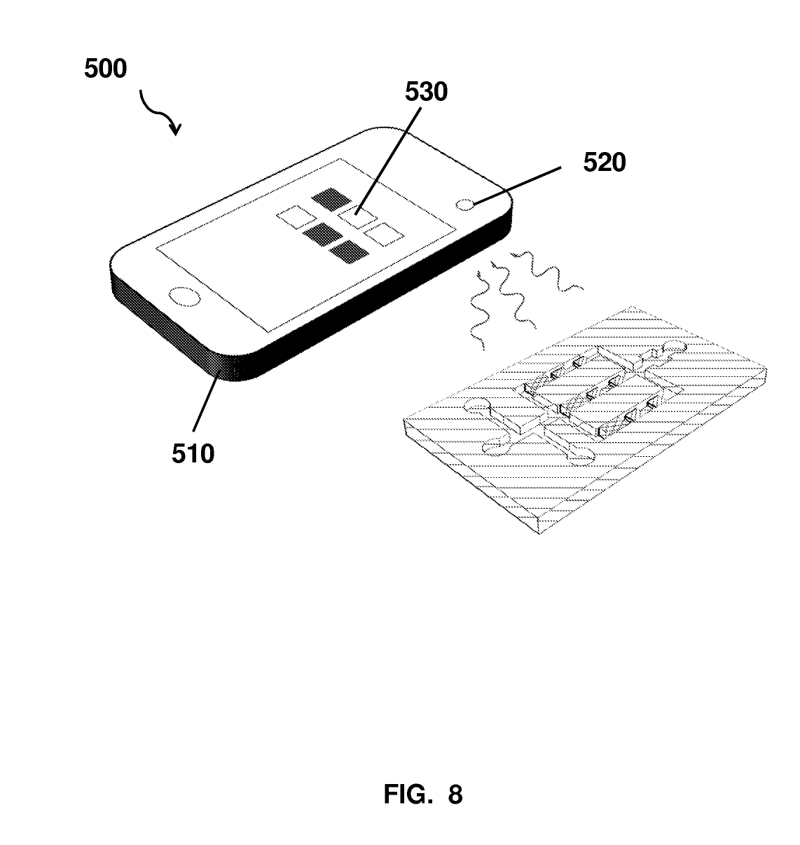

The detectors and the systems in the inventions can be adapted to a handheld system, such as a cell phone or smart phone (FIG. 8). The system has a size of hand held phone and the system can be entirely inside of the phone or a part of the phone. The microfluidic devices can be fabricated on a small cartridge with a size typical of today's SIM card from a cell phone. The integrated optical elements also have a dimension similar to the SIM card. (need to give dimension of the SIM card and detector) Light-emitting diodes (LED) can be used as an illumination source, as well as thin-film optical filters and CCD detectors. The entire system can fit inside a cell phone or be an independent chip carrier while using a cell phone's power, signal and computation capability. Also, the system can use a cell phone's remote communication system to send signals to hospitals, doctors and necessary locations, or even directly into a computer system. The entire device can be held by one hand preferably with the size of a human palm.

The advantage for being fast is because it has a very small cross-section and capture of analytes by capture of agent depends on the cross-section of the channel (give cross section of the channel). Furthermore, the microfluidic channel allows easy multiplexing to measure many different analytes at the same time. It also can help the integration of the entire system, which used to need to occupy desktop equipment, into a handheld or pocket size instrument. Such integration would drastically reduce the noise in the optical sensing and improve its signal. It also becomes very convenient to use and has low cost. It can be used together with cell phone and therefore can have more applications which are hard to be applied. It will allow more people at low cost and easier to use, convenient to use and allow more people to use the sensors. Therefore it is providing more ease of use. The low noise means that in the desktop system there may be noise coming from leaking lights, the vibration and light scattering the large system. By using integrated systems the noise can be eliminated because the low cost is coming from several aspects. One is that these chips can be fabricated in a monolithic way and at least part of the components can be fabricated monolithically and can be very inexpensive material. The use of the area also becomes much smaller in reduced cost.

The system is integrated with mobile telephone to process or communicate information obtained by the system. The integration means, the system (a) uses certain parts of smart phone's hardware or software, and/or (b) is built as a part of the phone, or partial built in the phone, or completely separately but still handheld.

### Assay Methods

The subject microfluidic device may be used to detect analytes in a sample. One method of sensing and/or quantifying an analyte or multiple analytes in a liquid comprises (a) having a microfluidic device 100; (b) flowing the liquid in a fluidic channel of the microfluidic device; (c) contacting the liquid with the capture agent on the nanosensor in the fluidic channel wherein the capture agent specifically binds to the analyte, and wherein the contacting is done under conditions suitable for specific binding of the analyte with the capture agent; and (d) reading a light signal from the analyte that is bound to or be in proximity of the capture agent. In the above step (a), before the bonding to the capture agent, the analyte may be labeled with a light-emitting label or not labeled (also referred as labeled directly or indirectly). In embodiments in which an analyte is no labeled with a light-emitting label before the bonding, the analyte, after the bonding to the capture agent, may be bound to a second capture agent (i.e. detection agent) (e.g., a secondary antibody or another nucleic acid) that is itself optically labeled, labeled secondary capture agent or labeled detection agent, (such process is also referred as indirectly labeling of an analyte). In a sensing using indirectly labeling, the labeled secondary capture agents unbounded to analytes are removed before the above reading step (b). In a sensing using directly labeling, the optical labels unbounded to analytes are removed before the above reading step (b).

In reading the light emitting labels on the assay, an excitation (photo, electro, chemical or combination of them) are applied to light emitting label, and the properties of light including intensity, wavelength, and location are detected.

In certain embodiments, the method comprises attaching a capture agent to the molecular adhesion layer of a subject nanosensors to produce a nanosensor, wherein the attaching is done via a chemical reaction of the capture agent with the capture agent-reactive group in the molecules on the molecular adhesion layer, as described above. Next, the method comprises contacting a sample containing a target-analyte with the nanosensor and the contacting is done under conditions suitable for specific binding and the target-analyte specifically binds to the capture agent. After this step, the method comprises removing any target-analytes that are not bound to the capture agent (e.g., by washing the surface of the nanosensor in binding buffer); Then detection agent conjugated with optical detectable label is added to detect the target-analyte. After removing the detection agent that are not bound to the target-analyte, The nanosensors can then be used, with a reading system, to read a light signal (e.g., light at a wavelength that is in the range of 300 nm to 1200 nm) from detection agent that remain bound to the nanosensor. As would be apparent, the method further comprises labeling the target analytes with a light-emitting label. This can be done either prior to or after the contacting step, i.e., after the analytes are bound to the capture agent. In certain embodiments, analytes are labeled before they are contacted with the nanosensor. In other embodiment, the analytes are labeled after they are bound to the capture agents of the nanosensor. Further, as mentioned above, the analyte may be labeled directly (in which case the analyte may be strongly linked to a light-emitting label at the beginning of the method), or labeled indirectly (i.e., by binding the target analytes to a second capture agent, e.g., a secondary antibody that is labeled or a labeled nucleic acid, that specifically binds to the target analyte and that is linked to a light-emitting label). In some embodiments, the method may comprise blocking the nanosensor prior to the contacting step (b), thereby preventing non-specific binding of the capture agents to non-target analytes.

The suitable conditions for the specific binding and the target-analyte specifically binds to the capture agent, include proper temperature, time, solution pH level, ambient light level, humidity, chemical reagent concentration, antigen-antibody ratio, etc.

In certain embodiments, a nucleic acid capture agent can be used to capture a protein analyte (e.g., a DNA or RNA binding protein). In alternative embodiments, the protein capture agent (e.g., a DNA or RNA binding protein) can be used to capture a nucleic acid analyte.

The sample may be a liquid sample and, in certain embodiments, the sample may be a clinical sample derived from cells, tissues, or bodily fluids. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate.

Some of the steps of an assay are shown in FIGS. 4 and 5. General methods for methods for molecular interactions between capture agents and their binding partners (including analytes) are well known in the art (see, e.g., Harlow et al., Antibodies: A Laboratory Manual, First Edition (1988) Cold spring Harbor, N.Y.; Ausubel, et al, Short Protocols in Molecular Biology, 3rd ed., Wiley & Sons, 1995). The methods shown in FIGS. 4 and 5 are exemplary; the methods described in those figures are not the only ways of performing an assay.

Some of the steps of an exemplary antibody binding assay are shown in FIG. 2. In this assay, nanosensors is linked to an antibody in accordance with the methods described above to produce a nanosensor 200 that comprises antibodies 202 that are linked to the molecular adhesion layer of the nanosensors. After nanosensor 200 has been produced, the nanosensor is contacted with a sample containing a target analyte (e.g., a target protein) under conditions suitable for specific binding. The antibodies 202 specifically bind to target analyte 204 in the sample. After unbound analytes have been washed from the nanosensor, the nanosensor is contacted with a secondary antibody 206 that is labeled with a light-emitting label 208 under conditions suitable for specific binding. After unbound secondary antibodies have been removed from the nanosensor, the nanosensor may be read to identify and/or quantify the amount of analyte 204 in the initial sample.

Some of the steps of an exemplary nucleic acid binding assay are shown in FIG. 3. In this assay, nanosensors 130 is linked to a nucleic acid, e.g., an oligonucleotide in accordance with the methods described above to produce a nanosensor 500 that comprises nucleic acid molecules 502 that are linked to the molecular adhesion layer. After nanosensor 200 has been produced, the nanosensor is contacted with a sample containing target nucleic acid 504 under conditions suitable for specific hybridization of target nucleic acid 504 to the nucleic acid capture agents 502. Nucleic acid capture agents 504 specifically binds to target nucleic acid 504 in the sample. After unbound nucleic acids have been washed from the nanosensor, the nanosensor is contacted with a secondary nucleic acid 506 that is labeled with a light-emitting label 508 under conditions for specific hybridization. After unbound secondary nucleic acids have been removed from the nanosensor, the nanosensor may be read to identify and/or quantify the amount of nucleic acid 504 in the initial sample.

One example of an enhanced DNA hybridization assay that can be performed using a subject device is a sandwich hybridization assay. The capture DNA is a single strand DNA functioned with thiol at its 3′-end The detection DNA is a single strand DNA functioned with a fluorescence label e.g., IRDye800CW at its 3′-end. Both the capture and detection DNA has a length of 20 bp. They are synthesized with different sequences to form complementary binding to a targeted DNA at different region. First the capture DNA is immobilized on the D2PA nanosensors's metal surface through sulfur-gold reaction. Then targeted DNA is added to the nanosensors to be captured by the capture DNA. Finally the fluorescence labeled detection DNA is added to the nanosensors to detect the immobilized targeted DNA. After washing off the unbound detection DNA, the fluorescence signal emanate from the nanosensors' surface is measured for the detection and quantification of targeted DNA molecules.

In the embodiments shown in FIGS. 2 and 3, bound analyte can be detected using a secondary capture agent (i.e. the “detection agent”) may be conjugated to a fluorophore or an enzyme that catalyzes the synthesis of a chromogenic compound that can be detected visually or using an imaging system. In one embodiment, horseradish peroxidase (HRP) may be used, which can convert chromogenic substrates (e.g., TMB, DAB, or ABTS) into colored products, or, alternatively, produce a luminescent product when chemiluminescent substrates are used. In particular embodiments, the light signal produced by the label has a wavelength that is in the range of 300 nm to 900 nm). In certain embodiments, the label may be electrochemiluminescent and, as such, a light signal can be produced by supplying a current to the sensor.

In some embodiments, the secondary capture agent (i.e. the detection agent), e.g., the secondary antibody or secondary nucleic acid, may be linked to a fluorophore, e.g., xanthene dyes, e.g. fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, IRDye800, IRDye800CW, Alexa 790, Dylight 800, etc.

The primary and secondary capture agents should bind to the target analyte with highly-specific affinity. However, the primary and secondary capture agents cannot be the molecule because they need to bind to different sites in the antigen. One example is the anti-human beta amyloid capture antibody 6E10 and detection G210, in which case 6E10 binds only to the 10^{th} amine site on human beta amyloids peptide while G210 binds only to the 40^{th} amine site. Capture agent and secondary capture agent do not react to each other. Another example uses rabbit anti-human IgG as capture antibody and donkey anti-human IgG as detection antibody. Since the capture and detection agents are derived from different host species, they do not react with each other.

Methods for labeling proteins, e.g., secondary antibodies, and nucleic acids with fluorophores are well known in the art. Chemiluminescent labels include acridinium esters and sulfonamides, luminol and isoluminol; electrochemiluminescent labels include ruthenium (II) chelates, and others are known.

### Applications

The subject methods and compositions find use in a variety applications, where such applications are generally analyte detection applications in which the presence of a particular analyte in a given sample is detected at least qualitatively, if not quantitatively. Protocols for carrying out analyte detection assays are well known to those of skill in the art and need not be described in great detail here. Generally, the sample suspected of comprising an analyte of interest is contacted with the surface of a subject nanosensor under conditions sufficient for the analyte to bind to its respective capture agent that is tethered to the sensor. The capture agent has highly specific affinity for the targeted molecules of interest. This affinity can be antigen-antibody reaction where antibodies bind to specific epitope on the antigen, or a DNA/RNA or DNA/RNA hybridization reaction that is sequence-specific between two or more complementary strands of nucleic acids. Thus, if the analyte of interest is present in the sample, it likely binds to the sensor at the site of the capture agent and a complex is formed on the sensor surface. Namely, the captured analytes are immobilized at the sensor surface. After removing the unbounded analytes, the presence of this binding complex on the surface of the sensor (i.e. the immobilized analytes of interest) is then detected, e.g., using a labeled secondary capture agent.

Specific analyte detection applications of interest include hybridization assays in which the nucleic acid capture agents are employed and protein binding assays in which polypeptides, e.g., antibodies, are employed. In these assays, a sample is first prepared and following sample preparation, the sample is contacted with a subject nanosensor under specific binding conditions, whereby complexes are formed between target nucleic acids or polypeptides (or other molecules) that are complementary to capture agents attached to the sensor surface.

In one embodiment, the capture oligonucleotide is synthesized single strand DNA of 20-100 bases length, that is thiolated at one end. These molecules are immobilized on the nanosensors' surface to capture the targeted single-strand DNA (which may be at least 50 bp length) that has a sequence that is complementary to the immobilized capture DNA. After the hybridization reaction, a detection single strand DNA (which can be of 20-100 bp in length) whose sequence are complementary to the targeted DNA's unoccupied nucleic acid is added to hybridize with the target. The detection DNA has its one end conjugated to a fluorescence label, whose emission wavelength are within the plasmonic resonance of the nanosensors. Therefore by detecting the fluorescence emission emanate from the nanosensors' surface, the targeted single strand DNA can be accurately detected and quantified. The length for capture and detection DNA determine the melting temperature (nucleotide strands will separate above melting temperature), the extent of misparing (the longer the strand, the lower the misparing). One of the concerns of choosing the length for complementary binding depends on the needs to minimize misparing while keeping the melting temperature as high as possible. In addition, the total length of the hybridization length is determined in order to achieve optimum signal amplification.

A subject sensor may be employed in a method of diagnosing a disease or condition, comprising: (a) obtaining a liquid sample from a patient suspected of having the disease or condition, (b) contacting the sample with a subject nanosensor, wherein the capture agent of the nanosensor specifically binds to a biomarker for the disease and wherein the contacting is done under conditions suitable for specific binding of the biomarker with the capture agent; (c) removing any biomarker that is not bound to the capture agent; and (d) reading a light signal from biomarker that remain bound to the nanosensor, wherein a light signal indicates that the patient has the disease or condition, wherein the method further comprises labeling the biomarker with a light-emitting label, either prior to or after it is bound to the capture agent. As will be described in greater detail below, the patient may suspected of having cancer and the antibody binds to a cancer biomarker. In other embodiments, the patient is suspected of having a neurological disorder and the antibody binds to a biomarker for the neurological disorder.

The applications of the subject sensor include, but not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

The detection can be carried out in various sample matrix, such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate.

In some embodiments, a subject biosensor can be used diagnose a pathogen infection by detecting a target nucleic acid from a pathogen in a sample. The target nucleic acid may be, for example, from a virus that is selected from the group comprising human immunodeficiency virus 1 and 2 (HIV-1 and HIV-2), human T-cell leukaemia virus and 2 (HTLV-1 and HTLV-2), respiratory syncytial virus (RSV), adenovirus, hepatitis B virus (HBV), hepatitis C virus (HCV), Epstein-Barr virus (EBV), human papillomavirus (HPV), varicella zoster virus (VZV), cytomegalovirus (CMV), herpes-simplex virus 1 and 2 (HSV-1 and HSV-2), human herpesvirus 8 (HHV-8, also known as Kaposi sarcoma herpesvirus) and flaviviruses, including yellow fever virus, dengue virus, Japanese encephalitis virus and West Nile virus. The present invention is not, however, limited to the detection of DNA sequences from the aforementioned viruses, but can be applied without any problem to other pathogens important in veterinary and/or human medicine.

Human papillomaviruses (HPV) are further subdivided on the basis of their DNA sequence homology into more than 70 different types. These types cause different diseases. HPV types 1, 2, 3, 4, 7, 10 and 26-29 cause benign warts. HPV types 5, 8, 9, 12, 14, 15, 17 and 19-25 and 46-50 cause lesions in patients with a weakened immune system. Types 6, 11, 34, 39, 41-44 and 51-55 cause benign acuminate warts on the mucosae of the genital region and of the respiratory tract. HPV types 16 and 18 are of special medical interest, as they cause epithelial dysplasias of the genital mucosa and are associated with a high proportion of the invasive carcinomas of the cervix, vagina, vulva and anal canal. Integration of the DNA of the human papillomavirus is considered to be decisive in the carcinogenesis of cervical cancer. Human papillomaviruses can be detected for example from the DNA sequence of their capsid proteins L1 and L2. Accordingly, the method of the present invention is especially suitable for the detection of DNA sequences of HPV types 16 and/or 18 in tissue samples, for assessing the risk of development of carcinoma.

In some cases, the nanosensor may be employed to detect a biomarker that is present at a low concentration. For example, the nanosensor may be used to detect cancer antigens in a readily accessible bodily fluids (e.g., blood, saliva, urine, tears, etc.), to detect biomarkers for tissue-specific diseases in a readily accessible bodily fluid (e.g., a biomarkers for a neurological disorder (e.g., Alzheimer's antigens)), to detect infections (particularly detection of low titer latent viruses, e.g., HIV), to detect fetal antigens in maternal blood, and for detection of exogenous compounds (e.g., drugs or pollutants) in a subject's bloodstream, for example.

The following table provides a list of protein biomarkers that can be detected using the subject nanosensor (when used in conjunction with an appropriate monoclonal antibody), and their associated diseases. One potential source of the biomarker (e.g., “CSF”; cerebrospinal fluid) is also indicated in the table. In many cases, the subject biosensor can detect those biomarkers in a different bodily fluid to that indicated. For example, biomarkers that are found in CSF can be identified in urine, blood or saliva, for example.

| Marker | disease |
|---|---|
| Aβ42, amyloid beta-protein (CSF) | Alzheimer's disease. |
| fetuin-A (CSF) | multiple sclerosis. |
| tau (CSF) | niemann-pick type C. |
| secretogranin II (CSF) | bipolar disorder. |
| prion protein (CSF) | Alzheimer disease, prion disease |
| Cytokines (CSF) | HIV-associated neurocognitive disorders |
| Alpha-synuclein (CSF) | parkinsonian disorders (neuordegenerative |
| | disorders) |
| tau protein (CSF) | parkinsonian disorders |
| neurofilament light chain (CSF) | axonal degeneration |
| parkin (CSF) | neuordegenerative disorders |
| PTEN induced putative kinase 1 (CSF) | neuordegenerative disorders |
| DJ-1 (CSF) | neuordegenerative disorders |
| leucine-rich repeat kinase 2 (CSF) | neuordegenerative disorders |
| mutated ATP13A2 (CSF) | Kufor-Rakeb disease |
| Apo H (CSF) | parkinson disease (PD) |
| ceruloplasmin (CSF) | PD |
| Peroxisome proliferator-activated receptor | PD |
| gamma coactivator-1 alpha (PGC-1α)(CSF) | |
| transthyretin (CSF) | CSF rhinorrhea (nasal surgery samples) |
| Vitamin D-binding Protein (CSF) | Multiple Sclerosis Progression |
| proapoptotic kinase R (PKR) and its | AD |
| phosphorylated PKR (pPKR) (CSF) | |
| CXCL13 (CSF) | multiple sclerosis |
| IL-12p40, CXCL13 and IL-8 (CSF) | intrathecal inflammation |
| Dkk-3 (semen) | prostate cancer |
| p14 endocan fragment (blood) | Sepsis: Endocan, specifically secreted by |
| | activated-pulmonary vascular endothelial |
| | cells, is thought to play a key role in the |
| | control of the lung inflammatory reaction. |
| Serum (blood) | neuromyelitis optica |
| ACE2 (blood) | cardiovascular disease |
| autoantibody to CD25 (blood) | early diagnosis of esophageal squamous cell |
| | carcinoma |
| hTERT (blood) | lung cancer |
| CAI25 (MUC 16) (blood) | lung cancer |
| VEGF (blood) | lung cancer |
| slL-2 (blood) | lung cancer |
| Osteopontin (blood) | lung cancer |
| Human epididymis protein 4 (HE4) (blood) | ovarian cancer |
| Alpha-Fetal Protein (blood) | pregnancy |
| Albumin (urine) | diabetics |
| albumin (urine) uria | albuminuria |
| microalbuminuria | kidney leaks |
| AFP (urine) | mirror fetal AFP levels |
| neutrophil gelatinase-associated lipocalin (NGAL) | Acute kidney injury |
| (urine) | |
| interleukin 18 (IL-18) (urine) | Acute kidney injury |
| Kidney Injury Molecule -1 (KIM-1) (urine) | Acute kidney injury |
| Liver Fatty Acid Binding Protein (L-FABP) (urine) | Acute kidney injury |
| LMP1 (saliva) | Epstein-Barr virus oncoprotein |
| | (nasopharyngeal carcinomas) |
| BARF1 (saliva) | Epstein-Barr virus oncoprotein |
| | (nasopharyngeal carcinomas) |
| IL-8 (saliva) | oral cancer biomarker |
| carcinoembryonic antigen (CEA) (saliva) | oral or salivary malignant tumors |
| BRAF, CCNI, EGRF, FGF19, FRS2, GREB1, and | Lung cancer |
| LZTS1 (saliva) | |
| alpha-amylase (saliva) | cardiovascular disease |
| carcinoembryonic antigen (saliva) | Malignant tumors of the oral cavity |
| CA 125 (saliva) | Ovarian cancer |
| IL8 (saliva) | spinalcellular carcinoma. |
| thioredoxin (saliva) | spinalcellular carcinoma. |
| beta-2 microglobulin levels - monitor activity of | HIV |
| the virus (saliva) | |
| tumor necrosis factor-alpha receptors - monitor | HIV |
| activity of the virus (saliva) | |
| CA15-3 (saliva) | breast cancer |

As noted above, a subject nanosensor can be used to detect nucleic acid in a sample. A subject nanosensor may be employed in a variety of drug discovery and research applications in addition to the diagnostic applications described above. For example, a subject nanosensor may be employed in a variety of applications that include, but are not limited to, diagnosis or monitoring of a disease or condition (where the presence of an nucleic acid provides a biomarker for the disease or condition), discovery of drug targets (where, e.g., an nucleic acid is differentially expressed in a disease or condition and may be targeted for drug therapy), drug screening (where the effects of a drug are monitored by assessing the level of an nucleic acid), determining drug susceptibility (where drug susceptibility is associated with a particular profile of nucleic acids) and basic research (where is it desirable to identify the presence a nucleic acid in a sample, or, in certain embodiments, the relative levels of a particular nucleic acids in two or more samples).

In certain embodiments, relative levels of nucleic acids in two or more different nucleic acid samples may be obtained using the above methods, and compared. In these embodiments, the results obtained from the above-described methods are usually normalized to the total amount of nucleic acids in the sample (e.g., constitutive RNAs), and compared. This may be done by comparing ratios, or by any other means. In particular embodiments, the nucleic acid profiles of two or more different samples may be compared to identify nucleic acids that are associated with a particular disease or condition.

In some examples, the different samples may consist of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

Although the foregoing embodiments have been described in some detail by way of illustration and example for purposes of clarity of understanding, it is readily apparent to those of ordinary skill in the art in light of the above teachings that certain changes and modifications can be made thereto without departing from the spirit or scope of the appended claims.

### EXAMPLES

Aspects of the present teachings can be further understood in light of the following examples, which should not be construed as limiting the scope of the present teachings in any way.

### Example I

### Fabrication and Demonstration of Ultra-Sensitive and Fast Fluorescence Immunoassay Using Novel Nanoplasmonic Sensor Inside Microfluidic Channels

The following example provides a description of fabrication and performances of a microfluidic device with a novel nanoplasmonic sensor integrated inside a microfluidic channel. The new assay has demonstrated 10⁶ fold detection limit enhancement of a model direct Protein A immunoassay over glass reference (from 2 nM to 850 aM, i.e. 120 ng/ml to 50 fg/ml) and 6 fold of incubation time reduction (2 hours to 20 min) compared to conventional 96-well plate immunoassay.

### Materials and Methods I

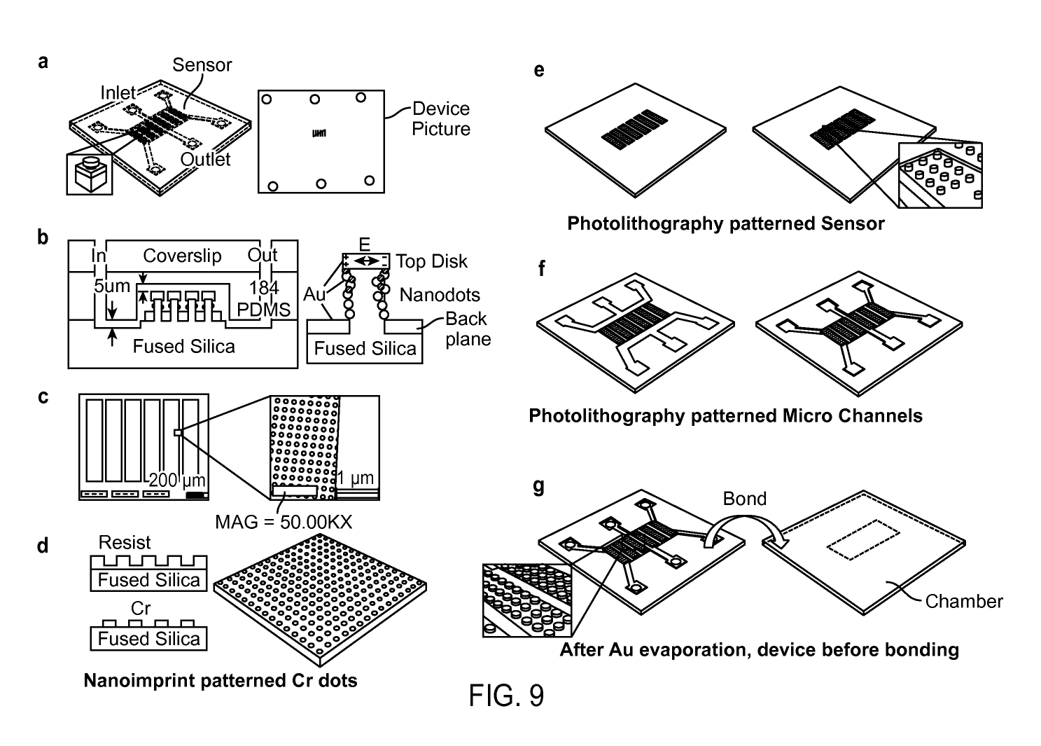

The assay has D2PA sensors in microfluidic channel (FIG. 9 panel A). The D2PA sensor consists of a dielectric nanopillar array (200 nm pitch, 70 nm diameter and 56 nm height) with an Au nanodisks on top of each pillar, an Au backplane on the foot, and random nanodots (5 nm to 15 nm) on the pillar sidewalls (FIG. 9 panel B). All metal components are self-aligned with each other and have nanogaps between them.

The fabrication of the microfluidic assay was done by three layer technology, where each layer are fabricated separately and then assembled. The three layers are: bottom D2PA sensor channel layer, middle PDMS inlet and outlet layer, and top thin glass cover layer (FIG. 9 panel B). To fabricate D2PA, Cr dots arrays were first patterned through nanoimprint. Nanopillars were then created through reactive ion etching (RIE) in photolithography defined regions. Then six shallow micro-channels (5 μm deep, 400 μm wide) and square reservoirs (5 μm deep, 500 μm wide) were fabricated by photolithography followed with RIE. Finally, 40 nm Au was evaporated on selected sensor area. Before sealing, the device was immersed in Dithiobis Succinimidyl Undecanoate (DSU) in 1,4 dioxane solution to coat a self-assembly monolayer (SAM) as capture agent for the immunoassay.

The PDMS inlet-and-outlet layer was fabricated through spinning and imprint^{[5]}. A PDMS film of 8 μm thick was first spin-coated onto a thin glass coverslip and then imprinted with a Si master mold. After imprinting, the PDMS was cured and then peeled off from the Si mold. Finally, the bottom layer with D2PA was aligned and bond to the PDMS/thin glass cover (FIG. 9 panels A-G).

For comparison, we also fabricated reference devices: (a) the microfluidic devices the same as D2PA microfluidic assay except no Au; for checking nanoplasmonic effects; and (b) D2PA plate on in 96-well plate (e.g. large fluid volume and no microfluidic channels) for checking microfluidic channel effects.

The increase of detection limit and the reduction of the assay incubation time of the microfluidic devices has been tested using a direct fluorescence immunoassay assay that detects fluorescence labeled (IRDye800CW) Protein A using the DSU monolayer as the capture agent. In the test, the labeled Protein A in PBS buffer solutions with concentrations from 1 fM to 100 nM (from 60 fg/ml to 6 mg/ml) of volume of 100 μL were separately injected into channels (one concentration per assay) using a flow rate of 5 μL/min. Protein A molecules were captured on the DSU SAM through the ester-amine reaction, the unbounded molecules were flushed out using 100 μL washer ((PBS+0.5% TWEEN-20) with a flow rate of 10 μL/min.

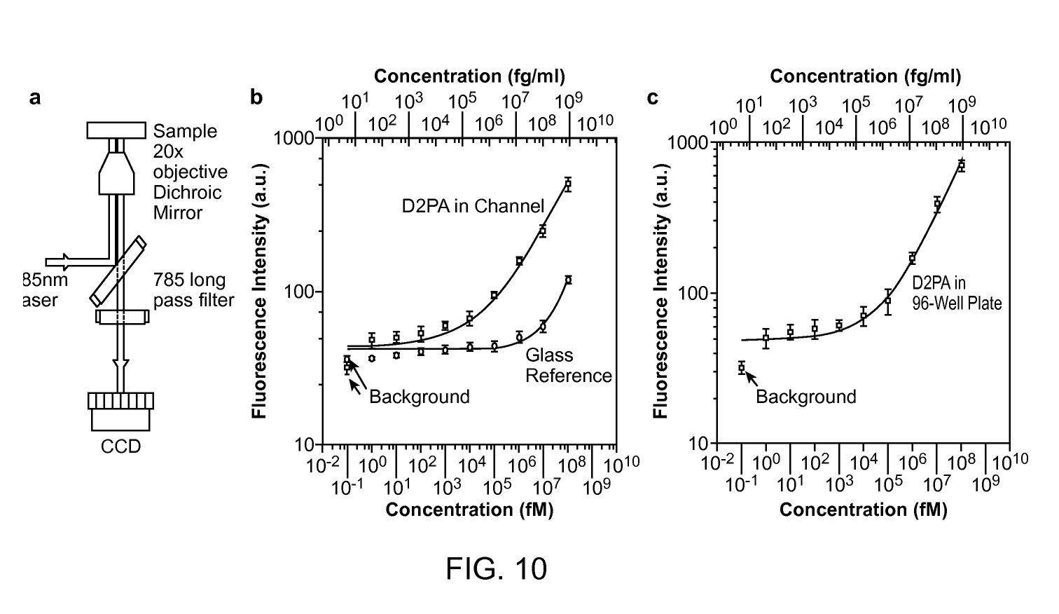

The assay in conventional 96-well plate reference was performed in a standard way: 100 μL labeled Protein A solutions were first added into separate wells and let it incubate for 2 hours. Then, each well was washed three times with washing solution. To read the immunoassay, fluorescence signal was collected through an inverted microscope equipped with an EM-CCD (FIG. 10 panel A) and averaged over an area of 100 μm by 100 μm. For each concentration, 5 replicates were measured to obtain the standard deviation.

### Results and Discussion I

A significant LoD enhancements and incubation time reduction in microchannel D2PA over the references have been observed. In fact, six orders of magnitude enhancement in detection sensitivity (LoD) was achieved on the new plasmonic device. FIG. 10 panel B shows the fluorescence intensity versus the Protein A concentration of the microchannel D2PA assay and the two references. Using the standard five-parameter logistic regression model, the microchannel D2PA assay demonstrated a limit LoD of 850 aM (50 fg/ml), which is about 10⁶ fold better than the identical assay on the reference sample without Au coating (LoD=2 nM). And the reference of D2PA in 96-well plate assay gave a LoD of 1 fM, similar to that of microchannel D2PA assay.

The improvement of limit of detection can be ascribed to two reasons: (1) the giant fluorescence enhancement of D2PA^{[8];} (2) the proper adhesive and spacer layer DSU, which captured Protein A and balanced the plasmonic enhancement and quenching effect of metallic structure.

The incubation time in the microchannel D2PA assay of the microfluidic assay has been found to be 6-fold shorter than D2PA in 96-well plate (from 2 hours to 20 min). The fast incubation is due to drastically reduce of average diffusion distance between the molecules to the capture layer which is D2PA surface.

### Example II

### Integration of Metallic Nanostructures in Fluidic Channels for Fluorescence and Rama Enhancement by Nanoimprint Lithography and Lift-Off on Compositional Resist Stack

The example provides a description of how to fabricate a microfluidic devices metallic nanostructures integrated into microfluidic channels, using nanoimprint lithography and lift-off on a compositional resist stack, which consists of multi-layers of SiO₂ and polymer patterned from different fabrication steps. The lift-off of the stack allows the final nano-features precisely aligned in the proper locations inside fluidic channels. The method provides high-throughput low-cost patterning and compatibility with various fluidic channel designs, and will be useful for fluorescence and Raman scattering enhancement in nano-fluidic systems.

### Materials and Methods II

The microfluidic device fabricated comprises micro-fluidic channels of different widths (2-8 μm) with nanoscale metal array inside the channel. The entire fabrication has three segments: (i) patterning micro-fluidic channels in fused silica substrate, (ii) patterning the nanoscale metal structures, and (iii) sealing the top of the microchannel using a slide. The step (ii) was done use a method, termed “lift-off using compositional-resist stack (LUCS)”, where the resists layers accumulated from several lithography steps in the fabrication form a 3D stack, which has all information regarding the size, area and alignment of nano-features. Therefore, when the metal nanostructures are lifted off by removing the resist stack, they are not only well-defined over a large area, but also accurately defined by the dimensions of the stacks and precisely aligned into the designed regions inside microchannels.

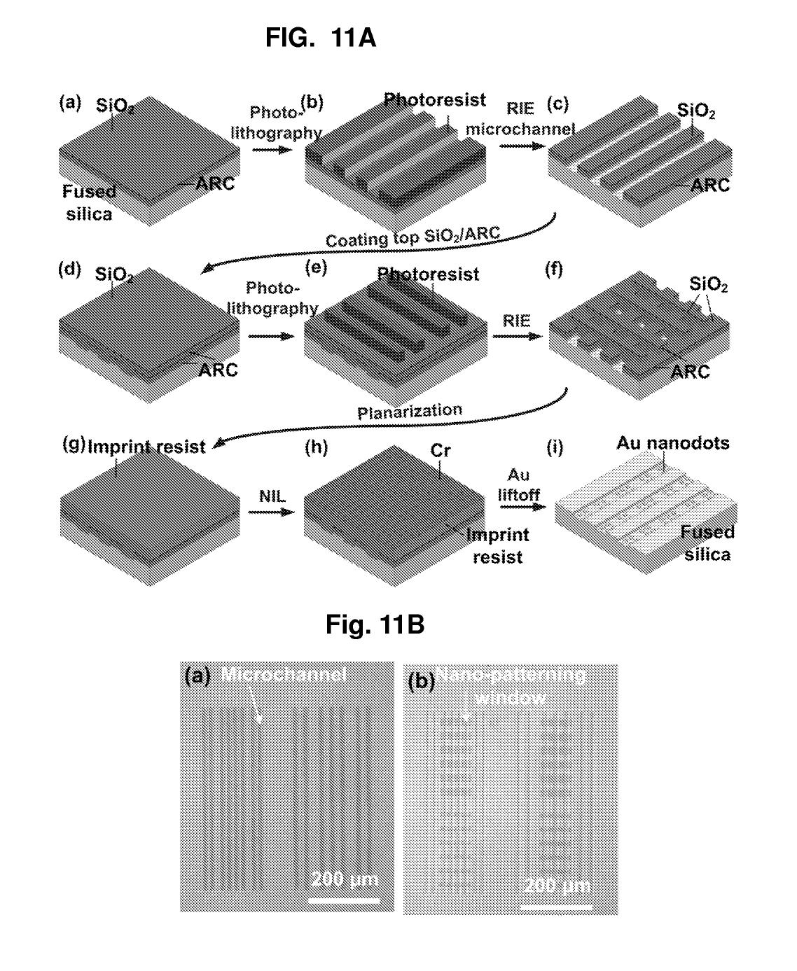

The major steps in LUCS are: (1) pattern micro-fluidic channels in fused silica substrate using SiO₂/ARC as the mask (FIG. 11 panels a-c), (2) without removing the remaining first SiO₂/ARC layer, spin-coat a second layer of SiO₂/ARC, and pattern the SiO₂ into a strip (FIG. 11 panels d-f), (3) spin-coat a third resist and imprint nanoholes in the resist, (4) transfer the nano-holes all the way to the fused silica through a Cr nano-mask (FIG. 11 panel g-h), (5) deposit and liftoff metal using the multilayer resist stack as the template to form the metal dots only in the desired locations in the microchannel (FIG. 11 panel i), (6) optionally etch the nanopillars in fused silica followed by other processing, and (7) seal the top with a glass plate.

Here the LUCS used a compositional mask of three resist stacks: the first two stacks each consist of SiO₂ and ARC (a crosslinked polymer similar to anti-reflection coating material, XHRiC-16 from Brewer Science, Inc.), and the third layer is the top imprint resist. To fabricate the microchannels bearing SiO₂/ARC stack, 1″ square fused silica wafers were thoroughly cleaned by solvents (acetone and 2-propanol) and RCA-1 (NH₄OH:H₂O₂:DI water=1:1:5, 80° C., 15 min), and then deposited with bottom-stack SiO₂/ARC (10/30 nm thick, ARC baked at 180° C. for 30 min). Photolithography (resist AZ 5214E, ˜1.4 μm thick) and reactive ion etching (RIE, Plasma Therm SLR 720) were then used to pattern SiO₂/ARC/fused silica, etching through the SiO₂/ARC stack and forming 50 nm deep channels in fused silica substrate. CHF₃ (10 sccm, 150 W, 5 mtorr), oxygen (10 sccm O₂, 50 W, 2 mtorr), and CF₄/H₂ (33/7 sccm, 300 W, 50 mtorr) were used in RIE for SiO₂, ARC, and fused silica, respectively. The photoresist was then solvent-stripped (FIG. 11B).

The middle-stack SiO₂/ARC (15/40 nm thick) layers were then deposited on the patterned fused silica wafers, and defined into rectangular openings crossing the fluidic channels by a second photolithography and RIE (FIG. 11A, d-e). The photoresist was then removed, exposing the selective nano-pattering windows in the middle SiO₂/ARC stack (FIG. 11, f). In this way, only the fluidic channel regions overlapped with the lithography-defined openings, where both the SiO₂/ARC stacks were etched away, would be patterned with nano-features.

To create uniform nano-features in NIL, the fused silica substrate was planarized with thermal imprint resist (Nanonex NXR-1025, 250 nm) by a flat Si mold (200 psi, 130° C., 5 min) in a nanoimprinter (Nanonex NX 2000) (FIG. 11, g). The flattened resist was imprinted to form 200 nm pitch nanohole arrays (200 psi, 130° C., 4 min), and then covered with 5 nm thick Cr nano-hole mask by shadow-evaporation (FIG. 11, h). Finally, the resist residual layer was removed by O₂ RIE, and Au/Cr nano-dots of 30/3 nm thick were nano-patterned in the channels (FIG. 11, i) by e-beam evaporation and lift-off in RCA-1 (80° C., 10 min). The fabricated device can then be treated with ozone and sealed with a clean fused silica coverslip.

### Results and Discussion II

The micro-channels of various widths (2-8 μm)) were fabricated using the above method. In NIL, a 200 nm-pitch nano-pillar mold of 15×15 mm² was used, with a pillar width of 60 nm and a height of 130 nm. Imprinted nano-holes were defined in the resist uniformly, covering the different micro-patterned regions over the whole wafer. As shown from the cross-sectional SEM image, the imprint resist filled faithfully inside the channels.

The liftoff of the compositional resist stack was carried out carefully in RCA1 solution, which dissolves the ARC layers and removes all the deposited metal dots on the top. After liftoff, 60 nm sized Au/Cr nano-dots of 30/3 nm thick were fabricated only in the fluidic channels. The thickness of the metal nano-dots was chosen small than the channel depth, hence guaranteeing the full inclusion of the nanostructures inside fluidics and providing a flat surface of the fluidic device for successful device bonding and reliable testing. The nano-dots were self-aligned in channels with different widths and further integrated into a fluidic system by patterning inlet and outlet in another photolithography and RIE. This demonstration shows our approach can provide fast and large-area nano-patterning inside fluidics, flexible integration of nano-structures to fluidic systems of various geometries, and a large tolerance in nano-scale multi-level alignment.

This LUCS approach can also be utilized to pattern non-metallic materials and/or fabricate other complicated nano-patterns, e.g. meshes, bars, and tri-angles, by simply using the corresponding imprint molds. For example, 115 nm diameter square fused silica nano-pillars were patterned by NIL using a different pillar mold and aligned in fluidic channels. Using the LUCS approach, functional and more complex nanostructures, such as plasmonic disk-coupled dots-on-pillar antenna array (D2PA), can also be fabricated in fluidic systems and used for real-time fluorescence and surface enhanced Raman (SERS) enhancement measurements.

Through the multi-level lithography steps and self-aligned integration, the proposed LUCS nano-patterning technique allows independent control of the geometries of the micro-channels (e.g. location, width, and depth) and the nano-dots (e.g. pitch, size, shape, thickness, and material), and thus enables the optimized flexible integration of nano-features into micro-fluidic systems. Because all the fabrication steps are standard techniques, tens or even hundreds of devices can be produced in a single batch, thus maximizing the throughput. Currently, the fabrication of a whole batch may take up to about 24 hours, mainly limited by the vacuum waiting time for evaporation. It is believed the fabrication time can be shortened by further optimization.

### Example III

### Enhanced Fluorescence Imaging of Lambda DNA Continuous Flow in a Nano-Opto-Fluidic System Integrated with Self-Aligned Nano-Plasmonic Structures

The following example provides a description of how to design, fabricate, and use a microfluidic device system with integrated plasmonic disk-coupled dots-on-pillar antenna arrays (D2PA), and how to use such device in real-time sensing the properties of a single DNA strand. The fabrication use a novel fabrication protocol, which uses self-aligned nanoimprint lithography for selective nano-patterning inside fluidic channels, optimized chip cleaning procedure to minimize surface roughness, and a room-temperature direct bonding technique for chip sealing. The D2PA geometry is optimized to achieve reliable chip integration, stretching of λ-DNA molecules, effective fluorescence enhancement (up to 30 times) of a high quantum-yield dye (0.46), and continuous DNA flow imaging.

### Materials and Methods III

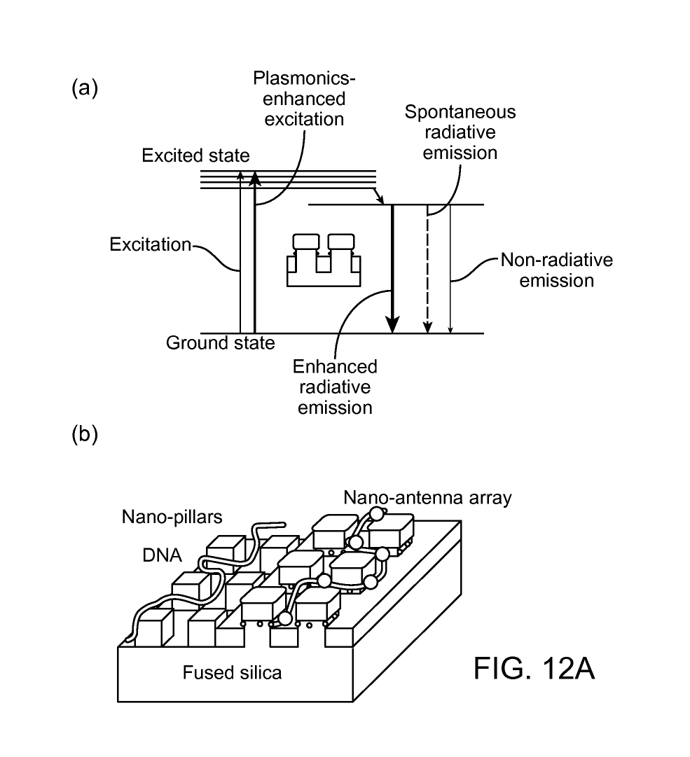

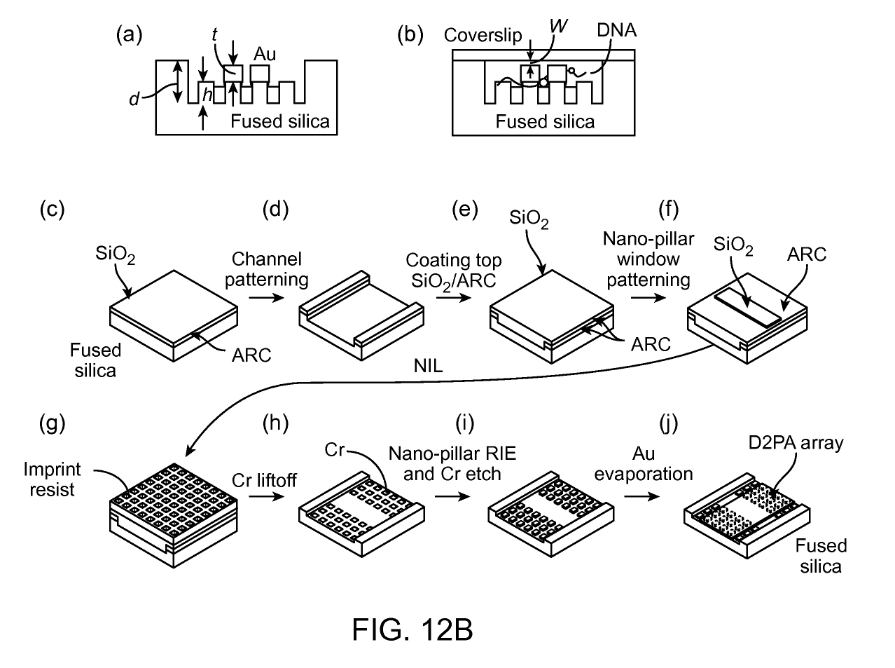

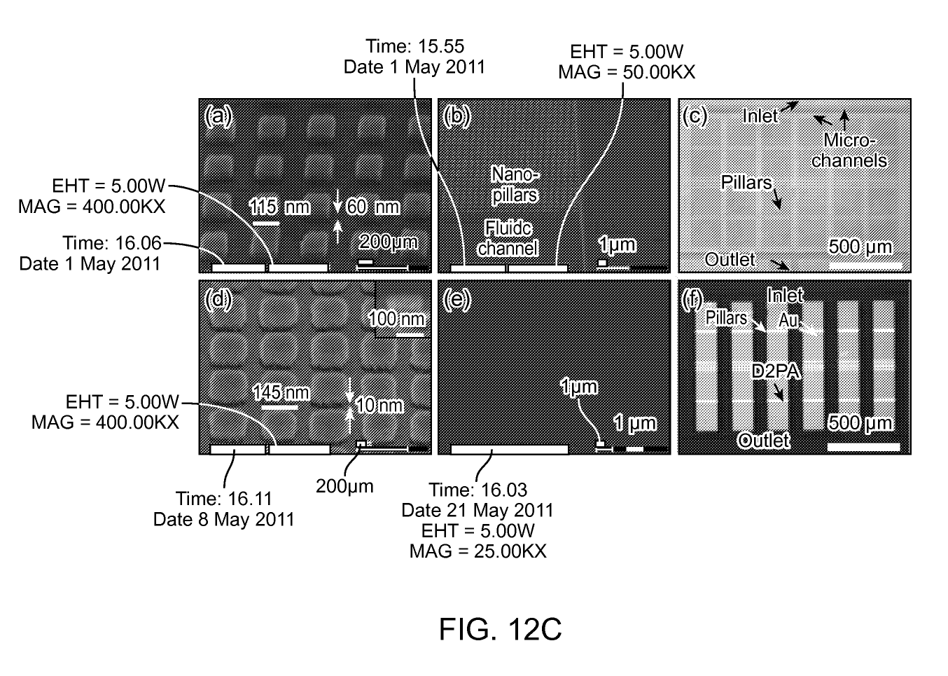

The microfluidic device has D2PA inside microfluidic channels. For successful integration into the fluidic chip, the geometry of the D2PA plasmonic structures has to be designed properly. Vertically, the fluidic channel depth d (=h+t+w, FIG. 12B, panel a) needs to be larger than the pillar height h plus gold thickness t. In this way, the D2PA Au disk top is kept a small gap w away from the coverslip to avoid bending the coverslip (FIG. 12B, panel b). Laterally, the plasmonic D2PA structures need to be defined only inside the channel so as not to cause surface roughness. This is achieved by a self-aligned NIL patterning approach.

The self-aligned NIL was achieved using a novel composite mask, which defines the nano-patterns in the target regions of fluidic channels and over the whole chip (FIG. 12B panels c-j). The composite mask uses three stacks, including bottom and middle stacks of SiO₂/ARC layers (ARC, a crosslinked polymer, commonly used as anti-reflection coating [17]) and a top nanoimprint resist layer, to pattern the fluidic channel, nano-patterning region, and nano-plasmonic structures, respectively. The reactive ion etching (RIE) process naturally self-aligns the SiO₂/ARC liftoff polymer masks to the channel edges, thus perfectly aligning the nano-patterns in channels.

The detailed chip fabrication includes the following steps. First, the bottom SiO₂/ARC stack, which consists of 40 nm thick spin-coated ARC (baked at 180° C. for 30 min) and 15 nm e-beam evaporated SiO₂, was coated on a 1″ square fused silica chip (FIG. 11B, c). Then photolithography and RIE (10 sccm CHF₃ at 100 W for SiO₂ and fused silica, and 10 sccm O₂ at 50 W for ARC) etched through the SiO₂/ARC layers and patterned the underlying fused silica into 200 μm wide, 1 mm long, 120 nm deep channels, self-aligning the SiO₂/ARC mask to the fused silica channel edges (FIG. 11B, d). After that, the middle stack of SiO₂/ARC (15/40 nm) was deposited on the chip (FIG. 11B, e), and this SiO₂ layer was patterned by another photolithography and CHF₃ RIE into isolated stripes crossing the channels (FIG. 11B, f) with the etching time carefully adjusted to minimize the attacking of ARC. Then the chip was UV-imprinted (150 PSI, 20° C., 4 min, UV 5 sec) using a nano-pillar mold (square pillar, 115 nm diameter, 200 nm pitch) to form nano-holes in the UV curable Si-free imprint resist (Nanonex NXR-2110, ˜200 nm thick) (FIG. 11B, g). UV NIL was preferred because the UV resist has a low viscosity and allows good resist filling on non-flat chip surface. After coating a Cr nano-hole mask onto the resist by a four-directional shadow evaporation, oxygen RIE was used to remove the residual layer in the nano-holes. Then 15 nm Cr was evaporated, and a 20 min RCA-1 (NH₃H₂O:H₂O₂:deionized water=1:1:5, 80° C.) cleaning stripped ARC polymer layers and also the Cr and SiO₂ layers on top, creating Cr nano-dot masks only in the fluidic channels and self-aligned to the channel edge (FIG. 11B, h). The Cr nano-dots masked CF₄/H₂ RIE to form 60 nm high nano-pillars, and then were etched by CR-7 etchant (FIG. 11B, i). The nano-pillar region was then connected to 700 nm deep inlet/outlet reservoirs and accessory micro-channels defined by photolithography and RIE, and access holes were aligned to the reservoirs and drilled through the chip by a sandblaster. Finally, the chip was thoroughly cleaned in solvents and H₂SO₄/H₂O₂ (1:1, 100° C., 1 hour), and deposited with 50 nm thick Au onto the nano-fused silica pillars, creating plasmonic D2PA antenna arrays in the fluidic channels (FIG. 11B, j). The device was further cleaned in solvents and ozone (15 min) and sealed with a fused silica coverslip for detection of DNA molecules (FIG. 11B, i-j).

Clearly seen from the scanning electron microscope (SEM) images, the NIL patterned nano-pillars into 60 nm high and 115 nm wide symmetric squares (FIG. 12A selectively self-aligned to the channels (FIG. 12C, b-c). After Au deposition, the Au D2PA antenna arrays had an enlarged diameter (˜145 nm) (FIG. 12, d) due to Au dewetting and diffusion on pillars, and self-assembled Au nano-dots also decorate on pillar sidewalls (FIG. 12C, d insert). The D2PA arrays were uniformly patterned with a large-area uniformity (FIG. 12, e), and defined as 150 μm wide regions in the six 200 μm wide fluidic channels (FIG. 12C, f) using photolithography, leaving 25 μm nano-pillar regions free from Au for fluorescence enhancement comparisons (FIG. 12C, f).

To reliably seal the fluidic chip, the fabrication, cleaning, bonding procedures were all carefully carried out. First, in fabrication the lateral self-aligned NIL patterning eliminates metal roughness outside of the channel, and the vertical channel depth (d=120 nm) is designed 10 nm larger than the total D2PA height (pillar height h=60 nm, gold thickness t=50 nm) to exclude coverslip bending in bonding. Second, the device surface was carefully protected, e.g. by photoresist (AZ 9260, ˜15 μm thick) and a blue tape (Semiconductor Equipment Corp.) in critical fabrication steps, such as access holes drilling and final Au deposition, to avoid sand debris or metal flakes on device surface which could cause a poor contact in bonding. Third, the drilled device was thoroughly cleaned by 1:1 mixed H₂SO₄/H₂O₂ (before final Au deposition) and ozone (after Au deposition), which removed any deposited polymer and dust particles and also increased the silanol group (—SiOH) density on the surfaces to facilitate a better bonding.

With the above issues carefully addressed, the 1″ square fluidic device was directly bonded at room temperature to a fused silica coverslip (0.17 μm thick, 24×24 mm²), which was treated by H₂SO₄/H₂O₂ and ozone. By gently pressing the two pieces together, an initial contact area was formed and immediately propagated to cover the whole device within less than 1 second, without trapping appreciable air bubbles in the device center. The device was then stored overnight at room temperature before being used for fluidic test. Room temperature direct wafer bonding is expected to yield a bonding strength of 0.1-0.2 J/m² [18-20], which although is much smaller than permanently bonding (˜2 J/m²) using a high-temperature (>1000° C.) annealing but allows a sufficiently long testing time (>24 hours in our test) for reliable fluidic manipulation. Since ozone cleaning was used instead of acids or high-temperature treatment, the Au plasmonic nano-structures were free from degradation, allowing the best D2PA performance.

After wafer bonding and overnight storage, the fluidic device was carefully mounted onto a home-made fluidic jig, which was used for loading running buffer and DNA and also connecting electrical supplies to the device. Then the device was loaded with 0.5×TBE buffer with 0.1 wt % POP6 (Applied Biosystems), electro-wetted at 10V for 8 hours to gently drive out all air bubbles in micro-channels, and then loaded with 30 μL DNA-containing 0.5×TBE buffer. The buffer included λ-DNA (48.5 kb, 16.5 μm, New England Biolabs Inc.) 5:1 labeled with POPO-3 fluorescence dye (Invitrogen Corp.), an oxygen scavenging system (3% β-mercaptoethanol, 4 mg/mL β-D-glucose, 0.2 mg/mL glucose oxidase, and 0.04 mg/mL catalase), and 10 mM anti-bleaching dithiothreitol (DTT). POPO-3 dye was used to label the DNA, because it is a stable intercalating dye with a high quantum yield (0.46), it has a low background noise (non-fluorescent when not bonded to DNA) and a high-sensitivity (>1000 times brighter when bonded to DNA), and its absorption (˜534 nm) well matches the resonance of the Au D2PA antenna arrays. The DNA imaging was performed on an inverted fluorescence microscope (Eclipse TE300, Nikon), with a green emission line of the mercury lamp at 546 nm used as the excitation source. DNA molecules were driven by electrophoresis (10 V) to flow from the micro-reservoirs into the D2PA-integrated fluidic channels, with the electric field applied ˜45° to the fluidic channel. An oil immersion objective (100×, 1.4 NA) was used for concentrating the illuminating light onto the sample and collecting fluorescence signals. The fluorescence signals passed through a 555 nm dichromatic beam-splitter and a band-pass filter (590/60, Nikon) before being recoded by an Andor Ixon3 electron multiplying charge-coupled device (EMCCD) camera.

Clearly, the DNA molecules accumulated at the edges of the fluidic channels as driven by the electric field, but no fluorescence signals were found outside the channels, showing evidently the device was well sealed. In fact, the room-temperature sealed device lasted for 24 hours without appreciable leakage, but it degraded faster afterwards and started to leak seriously 72 hours after wetting. After the testing, the device can be separated from the coverslip, rinsed with solvents and DI water, thoroughly cleaned by 1:1 mixed H₂SO₄/H₂O₂ to remove the deposited gold, polymer, and dust, and saved for future reuse.

To evaluate the plasmonic properties of the D2PA nano-structures, the reflection and transmission spectra of the D2PA sample were measured by a confocal spectrometer (LabRAM ARAMIS, HORIBA), with the signals collected by an Olympus objective (50×, NA 0.75). The absorption was calculated as unity deducted by the reflection and transmission. Clearly the D2PA structures had a much larger absorption (70%) compared to an Au film of the same thickness (18%) at a resonance wavelength of ˜548 nm, improving the absorption by >50% (i.e., D2PA-enhanced absorption, the D2PA absorption minus the Au film absorption). Such a short-wavelength resonance allowed the close match of the D2PA plasmonic resonance to the excitation light source (˜546 nm) and the absorption of intercalating dye POPO-3 (534 nm) for the best optical imaging performance.

To use the D2PA antenna arrays built in fluidic channels for continuously imaging, λ-DNA molecules intercalated with POPO-3 dye were electrophoretically driven to flow across the boundary of D2PA and fused silica nano-pillar regions, with the electric field applied with ˜45° to the boundary. The fluorescent images were captured consecutively with a duration of 0.3 second per frame. As seen from imaging, the DNA molecules in the nano-pillar region cannot be resolved but can be clearly seen in the D2PA regions, indicating greatly enhanced detection by the D2PA plasmonic structures. The average intensities were ˜2100 and ˜210 in the two regions with and without D2PA, making the average fluorescence enhancement factor of 10, in agreement with the DNA intensity values probed directly from the images. The maximum DNA fluorescence intensity in D2PA region was ˜6000, corresponding to an enhancement factor of about 30 at some specific hot-spots.

Such a great fluorescence enhancement of large λ-DNA molecules (48.5 kb, contour length ˜22 μm after staining [26], radius of gyration ˜0.73 μm) labeled with a high quantum-yield dye (0.46) has not been previously reported. There are several key reasons for such a high fluorescence enhancement in our fluidic system: (1) the D2PA structures under excitation (546 nm wavelength) can effectively boost electric field to better excite POPO-3 dye molecules (absorption peak 534 nm), and they can also reduce the fluorophore lifetime on the excited state and lead to more photon emission; (2) the large coiled DNA molecules (the radius of gyration, R_{g}˜0.73 μm]) were stretched by the sub 80 nm gaps between nano-structures (D2PA and nano-pillars) into linearized chains of 2 nm diameter [29], and thus can get closer to the hot spots where the electric field is concentrated; (3) the high density of hot spots (25 D2PA antennas each μm²), including the peripheral nano-gaps (10 nm vertically and 15-20 nm laterally) surrounding the Au disk antennas (field enhancement >200 times) and the vicinities (˜20 nm laterally and ˜40 nm vertically) of the edges of the Au disk antennas (field enhancement ˜10 times), provided high enhancement in electric field and fluorescence intensities; (4) the nano-confinement by the D2PA antennas (˜70 nm vertically from Au backplane to coverslip, and 60 nm between Au disks laterally) led to a large chance of effective dye excitation, e.g. ˜5% and ˜30% of finding the DNA in the hotspots of vertical cavity gaps and the Au antenna edges.

The averaged fluorescent image of 50 consecutive images shows clear DNA flowing traces with isolated bright spots in the D2PA region, but very weak signals in the nano-pillar region. This observation further confirmed the importance of plasmonic nanostructures to enhance molecular detection in fluidic channels. The bright fluorescent dots in the D2PA region correspond to the spots where the plasmonic fluorescence enhancement is the highest. Since the NIL patterned D2PA antenna arrays are expected to have very uniform geometrical and hence optical properties, such an effect can be mainly attributed to the spatial fluctuations of DNA (and hence the intercalating dye) during flow. As discussed previously, the linearized DNA molecules (2 nm diameter) flow through and randomly fluctuate in the nano-trenches (˜60 nm wide between Au disks) between the D2PA antennas, and as a result have different distances from the plasmonic hot-spots and thus different fluorescent enhancement efficiency. The DNA molecules close to the antenna edges or the vertical nano-cavity gaps are expected to achieve the most enhanced signals and display as bright spots. The continuous imaging by plasmonic D2PA structures was due to the high-density patterning of a pitch of 200 nm, which is very close to the resolution of the EMCCD camera (˜150 nm each pixel). By further reducing the D2PA pitch and enhance the hotspots density, we expect to achieve even brighter and more continuous DNA fluorescence signals.

What is claimed is:

1. A microfluidic device for detecting an analyte in a liquid, comprising:
a substrate;
a fluidic channel on a surface of the substrate; and
a nanosensor at a location of the channel, the nanosensor comprising:
i. a nanostructure, the nanostructure comprising at least one nanostructure element, each element comprising at least two metallic structures that are separated by a gap, and
ii. a capture agent deposited on a surface of the nanostructure,
wherein the capture agent specifically binds to the analyte; and
wherein the nanosensor amplifies a light signal to and/or from the analyte or a light label attached to the analyte, when the analyte is bound or in proximity to the capture agent.

2. The microfluidic device of claim 1, wherein the nanostructure element comprises:
i. a protrusion of a dielectric or semiconductor or a mix extending from a surface of a wall of the fluidic channel;
ii. a metallic cap on top of the protrusion; and
iii. a metallic back plane at the foot of the protrusion, the metallic back plane covering at least a portion of the fluidic channel wall surface near the foot of the protrusion and distanced from the metallic cap by a gap.

3. The microfluidic device of any prior claim, wherein the nanostructure element comprises:
i. a flat surface on a wall of the fluidic channel;
ii. a metallic back plane covering a portion of the flat surface;
iii. a protrusion of dielectrics or semiconductors on top of the metallic back plane, occupying a portion of the metallic back plane surface; and
iv. a metallic cap on top of the protrusion that is distanced from the metallic cap by a gap.

4. The microfluidic device of any prior claim, wherein each nanostructure element further comprises at least a metallic dot structure on sidewall of the protrusion, the metallic dot being distanced from the metallic cap or the metallic back plane by a gap.

5. The microfluidic device of any prior claim, wherein the top surface of the protrusion has a shape selected from the group of shapes consisting of round, triangle, polygonal, elliptical, elongated bar shaped, or any combination thereof.

6. The microfluidic device of any prior claim, wherein said metallic cap has substantially the same lateral geometry as the protrusion.

7. The microfluidic device of any prior claim, wherein the protrusion has a lateral dimension and/or a height less than the wavelength of said light.

8. The microfluidic device of any prior claim, wherein the lateral dimension of the metallic cap is in the range from 5 nm to 150 nm.

9. The microfluidic device of any prior claim, wherein the gap between the metallic cap and the metallic back plane is in the range of 0.1 nm to 60 nm.

10. The microfluidic device of any prior claim, wherein at least one of the metallic dot structures has dimensions in the range of 1 nm to 25 nm.

11. The microfluidic device of any prior claim, wherein the spacing between the two nearest protrusions of the plurality of elements is in the range from 2 nm to 200 nm.

12. The microfluidic device of any prior claim, wherein the thickness of the metallic cap and the metallic back plane is between 5 nm to 80 nm.

13. The microfluidic device of any prior claim, wherein the microfluidic channel is dimensioned such that the total depth of the fluid on the top surface of the nanosensor is in the range of 2 nm to 50 micron.

14. The microfluidic device of any prior claim, wherein the microfluidic channel has a cross-section such that the total fluid thickness on the top surface of the nanosensor is in the range of less than 500 micron.

15. The microfluidic device of any prior claim, wherein the metal is selected from the group consisting of gold, silver, copper, aluminum, platinum, alloys thereof, a semiconductor that exhibits plasmonic properties, and combinations thereof.

16. The microfluidic device of any prior claim, wherein the microfluidic device has more than one microfluidic channels.

17. The microfluidic device of any prior claim, wherein the fluidic channel has more than one nanosensors on different locations of (a) the same or different wall of the fluidic channel and/or (b) different fluid channels.

18. The microfluidic device of any prior claim, wherein the microfluidic device detects and/or quantifies more than one analytes in a liquid sample, simultaneously and/or sequentially, by having and using more than one nanosensor and/or more than one microfluidic channels on one microfluidic device.

19. The microfluidic device of any prior claim, wherein the microfluidic device further comprises an adhesion/spacer layer on the surface of the nanostructure, the adhesion/spacer layer links the surface with the capture agent,

20. The microfluidic device of claim 19, wherein the adhesion/spacer layer has a thickness of 0.5 nm to 50 nm and is selected to optimize the amplification of light signal.

21. The microfluidic device of claim 19, wherein the adhesion/spacer layer has a thickness of 0.1 nm to 10 nm and is selected to optimize the amplification of light signal.

22. The microfluidic device of claim 19, wherein the exterior surface of said molecular adhesion layer comprises a capture agent-reactive group, selected from an amine-reactive group, a thiol-reactive group, a hydroxyl-reactive group, an imidazolyl-reactive group and a guanidinyl-reactive group.

23. The microfluidic device of claim 22, wherein said capture agent-reactive group is a N-hydroxysuccinimidyl ester, sulfo-N-hydroxysuccinimidyl ester, a halo-substituted phenol ester, pentafluorophenol ester, a nitro-substituted phenol ester, an anhydride, isocyanate, isothiocyanate, an imidoester, maleimide, iodoacetyl, hydrazide, an aldehyde, or an epoxide.

24. The microfluidic device of any of claims 19-23, wherein said molecular adhesion layer is attached to the at least two metallic structures via a metal-sulfur bond.

25. The microfluidic device of any of claims 19-24, wherein the molecular adhesion layer is a monolayer of alkanethiol or thio-poly(ethylene) glycol.

26. The microfluidic device of any of claims 19-24, wherein the molecular adhesion layer is attached to the at least two metallic structures via a streptavidin/biotin interaction.

27. The microfluidic device of any of claims 19-25, wherein the exterior surface of said molecular adhesion layer comprises a biotin moiety or streptavidin.

28. The microfluidic device of any of claims 19-25, wherein the exterior surface of said at least two metallic structures comprises a streptavidin group that can bind to a biotinylated capture agent.

29. The microfluidic device of any prior claim, wherein the exterior surface of said at least two metallic structures comprises a biotin moiety that can bind to a streptavidin-linked capture agent.

30. The microfluidic device of any of claims 19-28, wherein, wherein said molecular adhesion layer is a self-assembled monolayer (SAM), wherein each molecule of the SAM comprises three parts: (i) a head group that has specific affinity to the metal surfaces of the nanodevice, (ii) a terminal group that specific affinity to the capture agent, and (iii) a linker that links the head group and terminal group, wherein the length of the linker determines the average spacing between the metal surfaces and an attached capture agent can affects light amplification of the nanodevice.

31. The microfluidic device of any prior claim, wherein said capture agent is a protein.

32. The microfluidic device of claim 31, wherein said capture agent is an antibody.

33. The microfluidic device of any prior claim, wherein said capture agent is a nucleic acid.

34. The microfluidic device of claim 33, wherein said capture agent is an oligonucleotide.

35. The microfluidic device of any prior claim, wherein the protrusion comprises a dielectric or semiconductor material selected from the group consisting of polymers, silicon-dioxide, silicon-nitride, hafnium oxide, aluminum oxide, silicon, gallium arsenide, and gallium nitride.

36. A system for detecting and/or quantifying an analyte in a liquid, comprising:
(a) a microfluidic device of claim 1;
(b) a holder for the microfluidic device;
(c) an excitation source for exciting a light signal from a label; and
(d) a reader adapted to for reading the light signal.

37. The system of claim 36, wherein the excitation source is a light source selected from a laser and a light emitting diode, an electrical source, or a chemical source.

38. The system of claim 36 or 37, wherein the reader is selected from a photodetector, a CCD camera, a CMOS camera, a spectrometer or an optical sensor, that is capable of producing a zero, one, two, or three dimensional information of the property of light from the nanosensor.

39. The system of any of claims 36-38, wherein the system is dimensioned to be used as a hand held device.

40. The system of claim 36, wherein the system is integrated with mobile telephone to process or communicate information obtained by the system.

41. A method for detecting and/or quantifying an analyte in a liquid, comprising:
(a) obtaining a microfluidic device of claim 1;
(b) flowing the liquid in a fluidic channel of the microfluidic device;
(c) contacting the liquid with the capture agent on the nanosensor in the fluidic channel wherein the capture agent specifically binds to the analyte, and wherein the contacting is done under conditions suitable for specific binding of the analyte with the capture agent; and
(d) reading a light signal from the analyte that is bound to or be in proximity of the capture agent.

42. The method of claim 41, wherein the detection and/or quantification of an analyte in a liquid sample is for diagnosing a disease or condition of a human, the liquid sample is obtained from the human, the analyte is a biomarker associated to the disease or condition, the capture agent on the nanosensor specifically binds to the biomarker, reading of a light signal from biomarker that remain bound to the capture agent indicates that the human has the disease or condition.

43. The method of claim 42, wherein the disease or condition is an infectious disease, a parasitic disease, an injuries, a cardiovascular disease, a cancer, a mental disorder, a neuropsychiatric disorder or an organic disease selected from a pulmonary disease and a renal disease.

44. The method of any of claim 41-43, wherein the liquid is obtained from food, the environment, or a human or non-human animal, wherein analyte is a marker associated with a condition, the capture agent specifically binds to the marker, and the method comprises reading a light signal from any marker molecules that remain bound to the capture agent indicates the condition of the subject.

45. The method of any of claims 41-44, wherein the methods include a) the detection, purification and quantification of an microorganism selected from a virus, fungus and bacteria from that has been isolated from water, soil, or a biological sample selected from a tissue or bodily fluid, or b) the detection, or quantification of chemical compounds or biological samples that pose hazard to food safety or national security selected from toxic waste, anthrax.

46. The method of any of claims 41-45, wherein the method further comprises labeling the analyte with a light-emitting label, either prior to or after the analyte is bound to the capture agent, the labeling is direct or indirect.

47. The method of claim 46, wherein the reading comprises: exciting the light emitting label and reading a light signal.

48. The method of claim 47, wherein the exciting is done using light, an electrical current, a chemical reaction, or any combination thereof.

49. The method of claim 48, wherein the light signal is selected from a group of luminescence, fluorescence, electroluminescence, chemiluminence, and combination thereof.

50. The method of claim 47, wherein the light signal is surface-enhanced Raman scattering.

51. The method of any of claims 41-50, comprising measuring at least one property of said light signal from the group of intensity, wavelength, and location of light.

52. The method of any of claims 41-51, wherein the analyte is a protein or nucleic acid.

53. The method of any of claims 41-51, wherein the analyte is a chemical compound.

54. The method of any of claims 41-53, wherein the capture agent has specific affinity for the analyte of interest and is selected from a group of a protein, an antibody, a nucleic acid, an oligonucleotide or an aptmer.

55. The method of any of claims 41-54, wherein the analyte is associated with a disease selected from cancer, neurological disease, cardiovascular disease, organic disease, an infectious disease and a parasitic disease.

56. The method of any of claims 41-54, wherein said label emits light at a wavelength in the range of 300 nm to 1200 nm.

57. The method of any of claims 41-54, wherein said method comprises blocking the nanosensor prior to the contacting step (c), thereby preventing non-specific binding of said capture agents to non-target analytes.

58. The method of claim 46, wherein the labeling is done by binding the analyte to a detection agent that specifically binds to the analyte and that is linked to a light-emitting label before the binding.

59. The method of claim 58, wherein said detection agent is a nucleic acid that is linked to a light-emitting label.

60. The method of any of claims 41-59, wherein said method comprises blocking said nanosensor prior to said contacting step (c), thereby preventing non-specific binding of said capture agents to non-target analytes.

61. The method of any of claims 41-60, wherein said liquid sample comprises amniotic fluid, aqueous humour, vitreous humour, whole blood, fractionated blood, plasma, serum, breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine or exhaled condensate.

62. The method of any of claims 41-61, wherein said sensor is employed to detect or quantify (i) chemical compounds or biomolecules that correlate with the stage of a disease, (ii) a microorganism, (iii) chemical compounds or biological entities that pose hazard to food safety or national security, (iv) a vital parameter in a medical or physiological monitor, (v) vital parameter is glucose, blood oxygen level, or total blood count, (vi) a specific DNA or RNA from a biosample, (vii) the sequence and compare genetic sequences in DNA in the chromosomes or mitochondria.

63. The method of claim 58, wherein said detection agent is a secondary antibody that comprises a light-emitting label.

64. The method of claim 58, wherein said labeled analyte is linked to said light-emitting label via a streptavidin/biotin interaction.

65. A method for fabricating the microfluidic device of claim 1, comprising:
(a) patterning at least one protrusion on a surface of a substrate, the protrusion occupies, after the patterning, a portion of the surface;
(b) depositing a metallic material layer to the top of the protrusion and an area of the surface that is not occupied by the protraction, where in the depositions occur in parallel;
(c) patterning a microfluidic channel around the protrusion, wherein the patterning is before or after, or partially before and partially after the protrusion patterning and the metal deposition;
wherein the protrusion and the metallic structures form the nanostructures of the microfluidic device in claim 1.

66. The method of fabrication of claim 65, wherein the deposition of metallic material further comprises depositing the same metallic material on protrusion sidewall in the same process as the deposition on the protrusion top and the open area of the surface, the same metallic material on sidewall of the protrusion.

67. The method of fabrication of claim 65, wherein the method of fabrication further comprises depositing a metallic layer on the surface before the patterning of the protrusion.

68. A method for fabricating the microfluidic device of claim 1, comprising:
(a) depositing and patterning a lift-off template layer on a surface of a substrate, the lift-off layer has a hole that exposing the substrate surface;
(b) depositing materials needed for the metallic structures and dielectric/semiconductor protrusion from the top of lift-off template, a portion of the deposited material is inside the hole and in contact with the substrate surface and a portion of the deposited materials is on top surface of the lift-off template and not directly in contact with the substrate surface;
(c) dissolving the lift-off template in a solution, wherein the materials deposited on the top of lift-off template is separated from the substrate and the materials deposited inside the hole is remain on the substrate.
(d) patterning a microfluidic channel around the protrusion, wherein the patterning is before or after, or partially before and partially after the dissolving of the lift-off template

69. The method of fabrication of any of claims 65-68, wherein the method of patterning comprises nanoimprint.

70. The method of fabrication any of claims 65-68, wherein the method of fabrication further comprises depositing a capture agent for sensing an analyte onto a nanosensor in the microfluidic device, wherein the deposition is either before or after the patterning of the microfluidic channel.

71. The method of fabrication of any of claims 65-68, wherein the patterning a microfluidic channel around the protrusion after the protrusion patterning and the metal deposition comprises (i) fabricating open microfluidic channels on anther substrate, (ii) bonding the substrate with a substrate with the protrusion and the metallic materials; wherein the substrates are aligned and the protrusion is inside a microfluidic channel after the bonding.
